(19) United States
(12) Patent Application Publication
(10) Pub. No.: US 2022/0152120 A1
(43) Pub. Date: May 19, 2022

(54) BIRTH TISSUE-DERIVED PRODUCTS AND PREPARATION AND USES THEREOF

(71) Applicant: LifeNet Health, Virginia Beach, VA (US)

(72) Inventors: Xiaofei Qin, Virginia Beach, VA (US); Fanwei Meng, Virginia Beach, VA (US); Jingsong Chen, Virginia Beach, VA (US)

(73) Assignee: LifeNet Health, Virginia Beach, VA (US)

(21) Appl. No.: 17/603,661

(22) PCT Filed: Apr. 16, 2020
(86) PCT No.: PCT/US2020/028604

§ 371 Date: Oct. 14, 2021

Related U.S. Application Data
(60) Provisional application No. 62/834,687, filed on Apr. 16, 2019.
(60) Provisional application No. 62/932,055, filed on Nov. 7, 2019.
(60) Provisional application No. 62/981,973, filed on Feb. 26, 2020.

(51) Int. Cl. A61K 35/50; A61K 35/51; A01N 1/02

(52) U.S. Cl. CPC A61K 35/50; A01N 1/0231; A61K 35/51

(57) ABSTRACT

The present invention provides a composition for treating a pathological condition in a body part of a patient in needed thereof, comprising an effective amount of a birth tissue elute or birth tissue particulates. A method for preparing the birth tissue elute, for example, an umbilical cord elute, is also provided. A method for treating a pathological condition in a body part of a patient in needed thereof is further provided.

### CROSS-REFERENCE TO RELATED APPLICATIONS

This application claims the benefit of U.S. Provisional Application Nos. 62/834,687, filed Apr. 16, 2019, 62/932,055, filed Nov. 7, 2019, and 62/981,973, filed Feb. 26, 2020, the contents of each of which are incorporated herein by reference in their entireties for all purposes.

### FIELD OF THE INVENTION

The invention relates birth tissue derived products such as birth tissue elutes, birth tissue particulates and placental membrane sheets, and preparation and uses thereof.

### BACKGROUND OF THE INVENTION

Arthritis is inflammation of one or more of joints. The most common types of arthritis are osteoarthritis and rheumatoid arthritis. Osteoarthritis (OA) is a joint disease affecting more than 25 million Americans and 240 million people globally. The most common symptoms of OA are pain and movement limitation that have a significant impact on quality of life and patients' social and economic activities. This common joint malady is characterized by progressive deterioration and loss of articular cartilage with concomitant structural and functional changes in the entire joint, including the synovium, meniscus (in the knee), periarticular ligaments, and subchondral bone. Inflammation of a synovial membrane or synovium is called “synovitis”, which manifests as synovial membrane thickening and/or joint effusion. The presence of synovitis in OA is associated with more severe pain and joint dysfunction. In addition, synovitis may be predictive of faster rates of cartilage loss in certain patient populations.

The current treatment for OA includes nonsteroidal anti-inflammatory drug (NSAID), intra-articular corticosteroid, intra-articular hyaluronic acid (HA), and other intra-articular treatments such as platelet rich plasma (PRP) or mesenchymal stem cells injection. Currently none of these treatments showed the capability to stop the progression of OA or reverse the damage caused. Treatments with NSAID, corticosteroid or HA injection are more focusing on the symptom relief for short period of time such as weeks to months. PRP is a biological and autologous therapy that uses the patient's own blood in order to obtain plasma with a higher platelet concentration than blood. However, besides the variability of each patient's health condition, a great variability exists in the PRP preparation protocols used by different clinicians that sometimes causes contradictory results. Similar dilemma was found in autologous mesenchymal stem cell therapy.

Birth tissues provide a good source of active biomolecules as well as abundance of extracellular matrix scaffold for cutaneous regenerative purposes. A human amniotic membrane has been used to wrap around tissues such as repaired tendons by acting as a natural surgical barrier to reduce scar formation and adhesion to the surrounding tissues. There remains a need for birth tissue-derived products suitable for delivery into a body part such as a joint or tissue for treating a pathological condition in the body part.

### SUMMARY OF THE INVENTION

The present invention provides birth tissue-derived products, including elutes and particulates of a birth tissue such as an umbilical cord and a placental membrane from amniotic sac, and sheets of a placental membrane from amniotic sac, and preparation and uses of the birth tissue-derived products.

A method for preparing an elute of a birth tissue. The elute preparation method includes mixing particulates of a birth tissue with a liquid to form a mixture, incubating the mixture, and collecting a supernatant from the mixture. The supernatant is an elute of the birth tissue. The ratio of the weight of the birth tissue particulates to the volume of the liquid is in the range from 1:1 to 1:100. The mixture is incubated at a temperature from −5° C. to 15° C. for 1-240 hours.

According to the elute preparation method, the birth tissue may be selected from the group consisting of an umbilical cord, an amniotic sac, a placental plate and a combination thereof. In one embodiment, the birth tissue may be an umbilical cord. In another embodiment, the birth tissue may be a placental membrane, which is derived from an amniotic sac. The placental membrane may comprise amniotic membrane, chorionic membrane and trophoblast layer. The birth tissue may not have been treated with an enzyme not from the birth tissue. The birth tissue may have an average surface area in the range of 1-2,500 cm². The birth tissue may comprise viable cells. The viable cells may be from the birth tissue. The birth tissue may not comprise viable cells. The birth tissue may have been cryopreserved. The birth tissue may have been lyophilized or frozen. The birth tissue may not have been treated with an enzyme not from the birth tissue.

According to the elute preparation method, the particulates may have an average particle size in the range of 10-2,000 μm. The particulates may comprise viable cells. The particulates may not comprise viable cells. The particulates may have been cryopreserved. The particulates may have been lyophilized or frozen. The particulates may not have been treated with an enzyme not from the birth tissue.

The elute preparation method may further comprise micronizing a processed birth tissue to make the particulates. The processed birth tissue may be micronized.

According to the elute preparation method, the processed birth tissue may be a processed umbilical cord. The processed umbilical cord may not comprise an umbilical artery. The processed umbilical cord may not comprise umbilical cord vein endothelial cells. The processed umbilical cord may have been cryopreserved. The processed umbilical cord may comprise viable cells. The processed umbilical cord may not comprise viable cells. The processed umbilical cord may have been lyophilized or frozen.

According to the elute preparation method, the liquid may be selected from the group consisting of a culture medium, conditioned medium, isotonic solution, hypotonic solution, and water. The liquid may be a culture medium.

According to the elute preparation method, the mixing step may be performed on a mixing device. The elute preparation method may not comprise using a detergent, surfactant or a combination thereof.

According to the elute preparation method, the elute may have a shear viscosity of 0.1-10 Pa·s at 0.5 Hz.

According to the elute preparation method, the elute may comprise hyaluronic acid (HA), which may be from the birth tissue. The elute preparation method may further comprise adjusting the concentration of the hyaluronic acid (HA) in the elute. The elute may not comprise hyaluronic acid (HA) not from the birth tissue.

According to the elute preparation method, the elute may comprise a cytokine, which may be from the birth tissue. The cytokine may be interleukin-1 receptor antagonist (IL-1RA), which may be from the birth tissue. The elute preparation method may further comprise adjusting the concentration of the cytokine in the elute. The elute may not comprise a cytokine not from the birth tissue.

According to the elute preparation method, the elute may comprise a growth factor, which may be from the birth tissue. The growth factor may be selected from the group consisting of basic fibroblast growth factor (bFGF or FGF2) and transforming growth factor beta (TGF-beta). The elute preparation method may further comprise adjusting the concentration of the growth factor in the elute. The elute may not comprise a growth factor not from the birth tissue.

According to the elute preparation method, the elute may comprise a protease inhibitor, which may be from the birth tissue. The elute may comprise a protease, which may be from the birth tissue. The elute may not comprise a protease inhibitor not from the birth tissue. The elute may not comprise a protease not from the birth tissue. The protease may be a trypsin, serine protease, cysteine protease, threonine protease, aspartic protease, or metalloprotease. The protease inhibitor may be a tissue inhibitor of metalloproteinase (TIMP). The elute preparation method may further comprise adjusting the concentration of the protease inhibitor, for example, the TIMP concentration, in the elute.

According to the elute preparation method, the elute may comprise extracellular vesicles, which may be from the birth tissue. The extracellular vesicles may be CD40+. The elute may not comprise extracellular vesicles not from the birth tissue.

According to the elute preparation method, the elute may comprise exosomes, which may be from the birth tissue. The exosomes may be CD9+. The elute may not comprise exosomes not from the birth tissue.

According to the elute preparation method, the elute may comprise less than 5 mg/ml solubilized collagen, which may be from the birth tissue. The elute may comprise less than 5 mg/ml solubilized laminin, which may be from the birth tissue.

The elute preparation method may further comprise adjusting the concentration of one or more bioactive components in the elute. The one or more bioactive components may be from the birth tissue and may be selected from the group consisting of hyaluronic acid (HA), cytokines, growth factors, tissue inhibitors of metalloproteinase (TIMPs), extracellular vesicles and exosomes.

The elute preparation method may further comprise lyophilizing the elute. The method may further comprise dehydrating the elute.

The elute preparation method may further comprise storing the elute at a temperature below 40° C.

For each elute preparation method of the present invention, a birth tissue elute prepared according to the method is provided.

An elute composition for treating a pathological condition in a body part of a patient in need thereof is provided. The elute composition comprises an effective amount of an elute of a first birth tissue and a pharmaceutically acceptable carrier.

A particulate composition for treating a pathological condition in a body part of a patient in needed thereof is provided. The particulate composition comprises an effective amount of particulates of a first birth tissue and a pharmaceutically acceptable carrier.

The elute or particulate composition may be injectable.

For the elute or particulate composition, the first birth tissue may be selected from the group consisting of an umbilical cord, an amniotic sac, a placental plate and a combination thereof. The first birth tissue may be an umbilical cord. The first birth tissue may be prepared according the elute preparation method of the present invention.

The elute or particulate composition may have a shear viscosity of 0.1-500 Pa·s at 0.5 Hz. The elute or particulate composition may comprise one or more bioactive components, which may be from the first birth tissue, for example, hyaluronic acid (HA); a cytokine, which may be interleukin-1 receptor antagonist (IL-1RA); a growth factor, which may be selected from the group consisting of basic fibroblast growth factor (bFGF or FGF-2) and transforming growth factor beta (TGF-beta); a protease inhibitor; a tissue inhibitor of metalloproteinase (TIMP); extracellular vesicles, which may be CD40+; exosomes, which may be CD9+; less than 5 mg/ml solubilized collagen; and/or less than 5 mg/ml solubilized laminin.

The elute or particulate composition may comprise viable cells. The viable cells may be from the first birth tissue. The elute or particulate composition may not comprise viable cells. The elute or particulate composition may be lyophilized and/or stored at a temperature below 40° C.

The elute composition may further comprise particulates of a second birth tissue. The second birth tissue may be selected from the group consisting of an umbilical cord, an amniotic sac, a placental plate and a combination thereof. The second birth tissue may be an umbilical cord. The second birth tissue may be a placental membrane, and the placental membrane may comprise amniotic membrane, chorionic membrane and trophoblast layer.

The elute composition may further comprise one or more bioactive factors. The one or more bioactive factors may be from the first birth tissue. The one or more bioactive factors may be selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, HA, extracellular vesicles and exosomes.

The elute of the first birth tissue may comprise one or more bioactive factors. The one or more bioactive factors may be from the first birth tissue. The one or more bioactive factors may be selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, and HA. The elute of the first birth tissue may comprise IL1-RA at a concentration greater than 0.5 ng/mL; TIMP-1 at a concentration greater than 10 ng/mL; HA at a concentration greater than 0.2 mg/mL; TIMP-3 at a concentration greater than 0.3 ng/mL; PRG-4 at a concentration greater than 0.2 ng/mL; α2-macroglobulin at a concentration greater than 4 μg/mL; pentraxin-3 at a concentration greater than 30 ng/mL; and/or TGF-beta3 greater than 1 ng/mL.

The elute composition may further comprise double stranded DNA. The double stranded DNA may be from the first birth tissue. The elute of the first tissue may comprise double stranded DNA at a concentration greater than 0.1 ng/mL.

The elute composition may further comprise extracellular vesicles. The extracellular vesicles may be from the first birth tissue. The elute of the first tissue may comprise greater than 10,000 extracellular vesicles per mL.

The elute composition may further comprise exosomes. The exosomes may be from the first birth tissue. The elute of the first tissue may comprise greater than 10,000 exosomes per mL. The first birth tissue and the second birth tissue may be from the same donor. The particulates of the second birth tissue may have an average particle size in the range of 10-2,000 μm. The particulates of the second birth tissue may comprise viable cells. The particulates of the second birth tissue may not comprise viable cells. The particulates of the second birth tissue may have been cryopreserved. The particulates of the second birth tissue may be lyophilized and/or stored at a temperature below 40° C. The elute of the first birth tissue may have been lyophilized and/or stored at a temperature below 40° C. The particulates may have been dehydrated.

In the particulate composition, the first birth tissue may be a placental membrane.

In the elute or particulate composition, the placental membrane of the first or second birth tissue may comprise a cellular layer, a reticular layer and a pseudo-basement membrane. The placental membrane may further comprise an amniotic membrane. The placental membrane may further comprise a trophoblast layer. The placental membrane may further comprise an amniotic membrane and a trophoblast layer. In other words, the placental membrane may be intact. The placental membrane may have a thickness of 50-800 μm. The placental membrane may have fenestration. The placental membrane may have liquid absorption of 90-99%. The placental membrane may have a DNA content at least 90% less than that of a control non-decellularized placental membrane. The placenta membrane particulates may have been decellularized. The placenta membrane particulates may not have been denatured. The placenta membrane particulates may comprise viable cells. The placenta membrane particulates may not comprise viable cells. The placenta membrane particulates may have been lyophilized and/or stored at a temperature below 40° C.

The elute or particulate composition may further comprise glycerol.

The elute or particulate composition may further comprise hyaluronic acid (HA) not from the first birth tissue, the second birth tissue or a combination of first birth tissue and second birth tissue. The elute or particulate composition may not comprise an alcohol, which may not be glycerol.

For the elute or particulate composition, the body part may be a joint or tissue. The joint may be selected from the group consisting of knee, shoulder, hip, elbow, wrist, finger, toe, and ankle joints. The joint may be a knee joint. The tissue may be selected from the group consisting of tendon, ligament, bursa, fascia, cartilage, muscle, connective tissue, dermis, synovium, and enthesis.

For the elute or particulate composition, the pathological condition may be selected from the group consisting of osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain, arthroplasty, and muscle pain. The pathological condition may be selected from the group consisting of osteoarthritis, bursitis and fasciitis. The pathological condition may be inflammation. The elute or particulate composition may remain at least 50% effective for at least 3 months.

A method for treating a pathological condition in a body part of a patient in need thereof is provided. The treatment method comprises administering to the body part of the patient an effective amount of the composition of the present invention or a placental membrane sheet. The composition may be injected into the body part.

According to the treatment method, the placenta membrane sheet may comprise a cellular layer, a reticular layer and a pseudo-basement membrane. The placental membrane sheet may further comprise an amniotic membrane. The placental membrane sheet may further comprise a trophoblast layer. The placental membrane sheet may further comprise an amniotic membrane and a trophoblast layer. In other words, the placental membrane may comprise an intact placental membrane.

According to the treatment method, the placenta membrane sheet may have a thickness of 50-800 μm. The placental membrane sheet may have fenestration. The placental membrane sheet may have liquid absorption of 90-99%. The placental membrane sheet may have a DNA content at least 90% less than that of a control non-decellularized placental membrane.

According to the treatment method, the body part is not on the surface of the patient. The body part may be a joint or tissue. The joint may be selected from the group consisting of knee, shoulder, hip, elbow, wrist, finger, toe and ankle joints. For example, the joint may be a knee joint. The tissue may be selected from the group consisting of tendon, ligament, bursa, fascia, cartilage, muscle, connective tissue, dermis, synovium, and enthesis. The tissue may be a soft tissue surrounding a joint. The pathological condition may be selected from the group consisting of osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain and muscle pain. The pathological condition may be selected from the group consisting of osteoarthritis, bursitis and fasciitis. The pathological condition may be inflammation. The pathological condition may be a degenerative tissue defect.

Where the body part has a cutaneous wound, the treatment method may further comprise applying the placental membrane sheet onto the wound. The treatment method may further comprise applying a porous soft tissue scaffold onto the wound after the placental membrane sheet is applied onto the wound.

Where the pathological condition is osteoarthritis or synovitis in a joint and the joint comprises an inflamed synovial tissue, the treatment method may comprise injecting the composition into the inflamed synovial tissue.

Where the pathological condition is osteoarthritis or synovitis in a joint and the joint has a wound after an inflamed synovial tissue is removed from the joint, the treatment method may comprise applying the placental membrane sheet onto the wound. The patient may have received an open join surgery. The patient may have received an arthroscopic joint surgery.

The treatment method may further comprise reducing adhesiveness of the body part.

The treatment method may further comprise improving healing of the body part. The healing may be tendon-to-bone healing.

The treatment method further comprise improving incorporation and acceptance of an implant into the body part. The implant may be selected from the group consisting of allografts, xenografts, silicone implant, metal implant, device implant, breast implant, pacemaker implant, microchip implant, drug delivery device implant, and internal monitor implant.

The treatment method may further comprise wrapping a tissue with the placental membrane sheet. The tissue may be selected from the group consisting of a nerve, a tendon, a ligament, a bone, a muscle and a combination thereof.

The treatment method may further comprise recellularization of the placental membrane sheet in the patient with cells.

The treatment method may further comprise growing cells in the placental membrane sheet. The treatment method may further comprise migrating cells in the placental membrane sheet. The treatment method may further comprise remodeling the placental membrane sheet by cells. The cells may be selected from the group consisting of synoviocytes, macrophages, fibroblasts and a combination thereof.

A composition comprising a soluble portion and a solid portion is provided. The soluble portion is from a first birth tissue. The solid portion comprises particulates of a second birth tissue. The first birth tissue may be selected from the group consisting of an umbilical cord, an amniotic sac, a placental plate and a combination thereof. The first birth tissue may be an umbilical cord. The first birth tissue may be a placental membrane, and the placental membrane may comprise amniotic membrane, chorionic membrane and trophoblast layer. The second birth tissue may be selected from the group consisting of an umbilical cord, an amniotic sac, a placental plate and a combination thereof. The second birth tissue may be an umbilical cord. The second birth tissue may be a placental membrane, and the placental membrane may comprise amniotic membrane, chorionic membrane and trophoblast layer. The first birth tissue and the second birth tissue may be the same. The solid portion may be covered by the soluble portion in the lyophilized form and/or hydrated form.

The soluble portion and the solid portion each may comprise double stranded DNA. The soluble fraction and the solid fraction each may comprise one or more bioactive factors. The one or more bioactive factors may be selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, HA, extracellular vesicles and exosomes.

A method for providing one or more bioactive factors to a body part of a patient in need thereof is provided. The method comprises administering to the body part of the patient an effective amount of a composition, which comprises a soluble portion and a solid portion according to the present invention. The method may further comprise release 5-50% of the one or more bioactive factors to the body part within 1 minute after the administration. The one or more bioactive factors may be selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, HA, extracellular vesicles and exosomes.

The method may further comprise releasing 5-50% of IL1-RA, HA, TIMP-1, TIMP-3, PRG-4, α2-Macroglobulin, PTX-3 and/or TGF-beta3 to the body part within 1 minute after the administration.

The method may further comprise releasing 5-50% of the one or more bioactive factors to the body part from 1 minute to 1 hour after the administration. The one or more bioactive factors may be selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, HA, extracellular vesicles and exosomes. Where the composition is lyophilized, the method may further comprise rehydrating the composition with a buffer or water before the administration.

### BRIEF DESCRIPTION OF THE DRAWINGS

FIG. 1 shows a human umbilical cord segment as dissected and two arteries removed from the umbilical cord segment.

FIG. 2 shows shear viscosity of five umbilical cord conditioned medium samples, each of which was prepared with an umbilical cord from a different donor. All samples exhibited a shear thinning phenomenon at high strains (>10%), indicating that the samples behaved like a liquid and flowed at higher shear. Four of the samples exhibited a plateau at low shear strains (<10%), indicating a Newtonian behavior at low shears (pseudo plastic behavior).

FIG. 3 shows metabolic activities of human synoviocytes plated at 3 different cell concentrations, (A) 6250, (B) 12500, or (C) 25000 cells/cm², and cultured in an umbilical cord conditioned medium prepared with umbilical cords from 5 donors at various concentrations for 24 hours.

FIG. 4 shows metabolic activities of human dermal fibroblasts plated at 3 different concentrations, (A) 12500, (B) 18750, or (C) 25000 cells/cm², and cultured in an umbilical cord conditioned medium prepared with umbilical cords from 3 donors at various concentrations for 24 hours.

FIG. 5 shows metabolic activities of (A) RAW cells plated at 25000 cells/cm² and (B) cultured in an umbilical cord conditioned media prepared with umbilical cords from 3 donors at various concentrations for 24 hours.

FIG. 6 shows TNF-alpha secretion by RAW 264.7 cells cultured in an umbilical cord conditioned medium prepared with umbilical cords from 3 donors. An LPS solution was added either one day after conditioned media treatment (A) or one day before conditioned media treatment (B). Both demonstrated reduction of TNF-alpha secretion with conditioned media treatment in a dose-dependent manner.

FIG. 7 shows cells outgrowing from a processed umbilical cord segment that has been cryopreserved for 8 days at −80° C.

FIG. 8 shows expansion of cells outgrowing from a cryopreserved processed umbilical cord after 1 day (left) and 3 days (right) using tissue culture flasks.

FIGS. 9A-D show flow cytometry of cells outgrowing from a processed umbilical cord without cryopreservation using mesenchymal stem cell markers showing positive results with markers for CD29, CD44, CD73, CD105 and CD166 but negative results with markers for CD14, CD31, CD34, CD45 and CD19.

FIGS. 10A-D show flow cytometry of cells out growing from a cryopreserved umbilical cord using mesenchymal stem cell markers showing positive results with markers for CD29, CD44, CD73, CD105 and CD166 but negative results with markers for CD14, CD31, CD34, CD45, and CD19. These results suggest that cryopreservation did not change the cell phenotype.

FIG. 11 shows formation of a 6 mg/ml umbilical cord conditioned medium hydrogel.

FIG. 12 shows the anti-inflammatory effects of the injectable birth tissue formulation. All three injectable birth tissue formulations effectively reduced the TNF-alpha secretion from LPS stimulated RAW cells. More than 95% TNF-alpha reduction was seen in the RAW cells treated by the formulation 1 and formulation 3 at both concentrations tested when compared to the formulation volume control group. Formulation 2 at 10 mg/mL and 5 mg/mL resulted in a dose-dependent RAW cell TNF-alpha reduction of 92.5% and 86.9%, respectively, when compared to the formulation volume control group.

FIG. 13 shows the proliferative effects of the injectable birth tissue formulations on primary human synoviocyte. All 3 formulation groups effectively induced primary human synovicoyte proliferation at a percentage of 212%, 166% and 197%, respectively, when compared to the media control group.

FIG. 14 shows the injectable birth tissue formulations inhibit the MMP1 enzyme activity. All three injectable birth tissue formulations effectively inhibited the MMP1 enzyme activity.

FIG. 15 shows shear viscosity measurement of injectable birth tissue formulations from a representable donor. (A) Formulation with umbilical cord elute consistently showed lower shear viscosity when compared to the formulation without umbilical cord elute. Umbilical cord elute was able to reduce the shear viscosity of the injectable birth tissue formulations. (B) An average of 38% reduction, from 3 donors, in shear viscosity from the formulation with umbilical cord conditioned medium was observed when compared to the formulation without umbilical cord conditioned medium at 50% shear strain.

FIG. 16 shows cohesiveness test of injectable birth tissue formulations. The results showed that, at a concentration of 40 mg (dry) particulate/mL and above, the undiluted umbilical cord elute was able to enhance the cohesiveness of the injectable PM particulates.

FIG. 17 shows MMP1 enzyme activity inhibition by the injectable birth tissue formulations. (A) Injectable birth tissue formulations inhibited MMP1 enzyme activity. (B) The injectable birth tissue formulation with umbilical cord elute demonstrated significantly superior inhibitory effect than the injectable birth tissue formulation without umbilical cord elute at minute 35.

FIGS. 18A-B show time course biochemical factors release assay of injectable birth tissue formulations. Injectable birth tissue formulation with umbilical cord elute contained more readily available soluble biochemical factors at both 5 mins and 60 mins following rehydration when compared to the formulation without umbilical cord elute. Data from one representative donor is shown for each analyte.

FIG. 19 shows recombinant FGF-2 protection by umbilical cord elute. Both lyophilized and reconstituted umbilical elute (A and B) and frozen umbilical cord elute (C and D) showed protective effects of commercially available recombinant FGF-2 over heat degradation.

### DETAILED DESCRIPTION OF THE INVENTION

The present invention relates to birth tissue-derived products such as birth tissue elute, birth tissue particulates and placental membrane sheets, as well as their preparation and uses. The invention is based on a surprising discovery that a viscous supernatant of a culture medium used to incubate birth tissue particulates such as umbilical cord particulates, also referred to as an umbilical cord elute, showed unexpected anti-inflammatory effects, protease inhibition effect, cohesiveness enhancement effect, and biochemical factor shelf-life extension effect. The inventors have also discovered injectable compositions comprising the umbilical cord elute and particulates of an umbilical cord and/or a placental membrane, especially having intact amniotic membrane, chorionic membrane and trophoblast layer. The elute in the injectable composition provided sufficient concentration of soluble bioactive factors (e.g., biochemical factors) around birth tissue particulates and became functional immediately after hydration and/or application to a liquid environment. The present invention provides a more standardized therapy by combining the benefits of HA treatment and active biomolecule treatment, while the quantity of bioactive components can be tailored to better fit each patient's needs. In addition, this invention provides a new treatment that reconstructs inflamed synovium and provides more sustainable release of bioactive molecules to treat synovitis, whose only currently available treatment is synovectomy, the removal of the inflamed synovium.

The term “birth tissue” used herein refers to amniotic sac, umbilical cord, placental plate or a combination thereof. The “birth tissue-derived product” used herein refers to an elute, particulates or a sheet of a birth tissue, or a combination thereof.

The term “amniotic sac” as used herein refers to a thin but tough placental membrane that holds amniotic fluid in which an embryo and later a fetus develops. The amniotic sac comprises an inner layer (i.e., an amnion layer) and an outer layer (i.e., a chorion layer), The amnion layer comprises several sub-layers, for example, epithelium, a basement membrane, a compact layer, a fibroblast layer, and a spongy layer (from inside to outside). Similarly, the chorion layer comprises several sub-layers, for example, a cellular layer, a reticular layer, a pseudo-basement membrane, and a trophoblast layer (from inside to outside). A chorion membrane includes the cellular layer, the reticular layer and the pseudo-basement membrane. The amnion layer and the chorion layer each comprise cells as well as cellular and extracellular molecules (e.g., growth factors, enzymes, and extracellular matrix molecules). The amniotic sac may be obtained from a donor. The donor may be a mammal, for example, human, bovine, porcine, murine, ovine, equine, canine, caprine and feline, preferably a human.

The term “placental membrane” used herein refers to the tissue derived from amniotic sac, which include amnion layer (also known as amniotic membrane), chorion layer that include a cellular layer, a reticular layer, a pseudo-basement membrane, and trophoblast layer.

The terms “intact placental membrane” and “intact amnion/chorion layer” are used herein interchangeably, and refer to a tissue having an amnion layer and a chorion layer, including a cellular layer, a reticular layer, a pseudo-basement membrane, and a trophoblast layer, from amniotic sac without removal (e.g., separation and isolation) of any one or more of the amniotic membrane, the cellular layer, the reticular layer, the pseudo-basement membrane and the trophoblast layer from an intact placental membrane.

In some embodiments, the birth tissue may be an intact placental membrane, comprising an amniotic membrane, a cellular layer, a reticular layer, a pseudo-basement membrane and a trophoblast layer. In other embodiments, the birth tissue may be a placenta membrane obtained after one or more of an amniotic membrane, a cellular layer, a reticular layer, a pseudo-basement membrane and a trophoblast layer are removed from an intact placental membrane. The placental membrane according to this present invention may comprise the cellular layer, the reticular layer and the pseudo-basement membrane after the amniotic membrane and the trophoblast layer are removed from an intact placental membrane.

The placental membrane according to this present invention may comprise the amniotic membrane, the cellular layer, the reticular layer and the pseudo-basement membrane after the trophoblast layer is removed from an intact placental membrane.

The placental membrane according to this present invention may comprise the cellular layer, the reticular layer, the pseudo-basement membrane and the trophoblast layer after the amniotic membrane is removed from an intact placental membrane.

The term “particulates” as used herein refers to small pieces of a birth tissue. The particulates may have an average particle size in the range of 0.1-10,000, 0.1-5,000, 0.1-2,000, 0.1-1,000, 0.1-500, 0.1-100, 0.1-10, 0.1-1, 0.5-10,000, 0.5-5,000, 0.5-2,000, 0.5-1,000, 0.5-500, 0.5-100, 0.5-10, 0.5-1, 1-10,000, 1-5,000, 1-2,000, 1-1,000, 1-500, 1-100, 1-10, 5-10,000, 5-5,000, 5-2,000, 5-1,000, 5-500, 5-100, 5-10, 10-10,000, 10-5,000, 10-2,000, 10-1,000, 10-500, 10-100, 50-10,000, 50-5,000, 50-2,000, 50-1,000, 50-500, 50-100, 100-10,000, 100-5,000, 100-2,000, 100-1,000 or 100-500 μm. For example, the particulates may have an average particle size in the range of 10-2,000 μm.

The term “cryopreserved” or “cryopreserving” used herein refers to preserving a birth tissue or a product derived from a birth tissue such as an elute, particulates or a sheet of the birth tissue in a cryopreservation medium by cooling down the birth tissue or a product derived from the birth tissue below the freezing point of water.

The term “micronize” or “micronizing” used herein refers to cutting a piece of birth tissue into particulates. The tissue may be micronized mechanically by, for example, grinding, milling, chopping, pulverizing, or crushing.

The term “solubilized extracellular matrix components” used herein refers to extracellular matrix proteins in solution. The extracellular matrix proteins may be selected from the group consisting of collagen, hyaluronan/hyaluronic acid, laminin, and/or fibronectin, and a combination thereof.

The term “injectable composition” used herein refers to a composition that is suitable for delivery into a body part of a subject, for example, a patient, by injection. An injectable composition may be delivered via a needle, a cannula, or a catheter connected to a syringe or other delivery device. The injectable composition may have a small particle size in the range of 1-2000 micron. The composition may be injectable through 10-30 gauge needle.

The term “an effective amount” refers to an amount of a birth tissue, a birth tissue-derived product or a composition thereof required to achieve a stated goal (e.g., treating a pathological condition in a body part, reducing adhesiveness of a body part, improving healing of a body part, improving incorporation of an implant into a body part). The effective amount of a birth tissue, a birth tissue-derived product or a composition thereof may vary depending upon the stated goals and the physical characteristics of the composition.

The terms “solubilized” and “soluble” are used herein interchangeably and refer to bioactive components, bioactive factors or biochemical factors dissolved in a solvent, especially water. The resulting solution is homogeneous without separation of phases or layers. No precipitation is observed by naked eyes.

The term “adhesiveness of a body part” used herein refers to the likelihood for an object to attach to the surface of a body part. Reduction of adhesiveness of a body part prevents attachment of unwanted objects to the surface of the body part.

The term “healing of a body part” used herein refers to the process of reducing or mitigating a pathological condition in a body part. The healing of the body part may be evidenced by, for example, an increase or decrease in expression of one or more biomarkers known to be associated with the pathological condition. For example, the biomarker may be tumor necrosis factor-alpha (TNF-alpha), interleukin 1a, or interleukin 1b associated with inflammation, may be lubricin/proteoglycan 4 associated with synoviocyte activity.

The term “incorporation of an implant into a body part” used herein refers to integration of an implant into a body part as evidenced by the inclusion of the implant as part of a whole body part, or the body does not reject the implant by, for example, generating a thick encapsulation around the implant.

The term “a pathological condition” used herein refers to a condition in a body part, whether associated with a disease or not. The pathological condition may be related to a pathologic fracture, a pathologic tissue, or a pathologic process. Examples of pathological conditions include osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain and muscle pain.

The term “in vivo sustainable” used herein refers to the capability of a composition to remain effective, for example, for treating a pathological condition in a body part or joint, over a pre-determined time period. The composition of the present invention may remain at least 10, 20, 30, 40, 50, 60, 70 or 80% effective for a predetermined time period, which may be at least 0.5, 1, 2, 3, 4, 5 or 6 months or 1-2, 1-3 or 1-6 months.

The term “decellularization” or decellularize” used herein refers to removal of cells from a birth tissue or a birth tissue-derived product. The term “recellularization” used herein refers to addition of cells into a birth tissue or a birth tissue-derived product that has been decellularized.

The term “remodeling a placental membrane sheet” or “remodeling of a placental membrane sheet” used herein refers to a structural change of a placental membrane sheet, including structural reorganization, alteration, or renewal of a placental membrane sheet. The term “reorganization” as used herein refers to rearrangement of matrix components orientation, density, or ratio. The term “alternation” as used herein refers to change. The term “renewal” as used herein refers to replacement of old components by new components. Remodeling of a placental membrane sheet may be evidenced by cell growth in the placental membrane sheet, for example, outgrowth of cells from the placental membrane sheet, or migration of cells in the placental membrane sheet.

The term “recipient cells” used herein refers to the cells in a subject, for example, a patient, receiving a birth tissue-derived product such as a placental membrane sheet. The recipient cells may attach to the placental membrane, grow into the placental membrane and/or migrate in the placental membrane. Examples of recipient cells include fibroblasts, endothelial cells, stem cells, keratinocytes, macrophages, synoviocytes, chondrocytes, tenocytes, myoblasts, myocytes, progenitor cells, and epithelial cells.

The term “liquid absorption” used herein refers to uptake of a liquid by a birth tissue or a birth tissue-derived product. The absorbed liquid may comprise biological molecules and/or chemical compounds.

The term “porous soft tissue scaffold” or “porous sponge-like structure” used herein refers to a three-dimensional structure that is porous, elastic, flexible, fibrous, and resilient. In addition, the preferred “porous sponge-like structure” is substantially coherent (or cohesive) in the sense of holding together or staying substantially intact. As used herein, the terms “coherent” or “cohesive” refer to the property that the elements of the structure of a material are maintained substantially intact (in the sense of holding together rather than becoming disassembled or separated). For example, a cohesive or coherent injectable birth tissue formulation holds together and maintain the shape following injection into a liquid. In a dry state, the porous sponge-like scaffold of the present invention may quickly absorb fluid. In the wet state, the porous sponge-like scaffold of the present invention may maintain the porosity, cohesiveness, and/or integrity. The wet porous sponge-like structure may resist certain tensile stress, and bounce back and reabsorb fluid after being released from compression.

The present invention provides a method of preparing an elute of a birth tissue. The preparation method comprises mixing particulates of a birth tissue with a liquid to form a mixture, incubating the mixture, and collecting a supernatant from the mixture. The supernatant is an elute of the birth tissue, also called a birth tissue elute or a conditioned medium. Pieces of the same birth tissue or different birth tissues may be used.

The birth tissue may be an umbilical cord, amniotic sac, placental plate and a combination thereof. In one embodiment, the birth tissue may be an umbilical cord. In another embodiment, the birth tissue may be a placental membrane, which is derived from an amniotic sac. The placental membrane may comprise a cellular layer, a reticular layer and a pseudo-basement membrane. The placental membrane may comprise an amniotic membrane, a cellular layer, a reticular layer and a pseudo-basement membrane. The placental membrane may comprise a cellular layer, a reticular layer, a pseudo-basement membrane and a trophoblast layer. The placental membrane may comprise an amniotic membrane, a cellular layer, a reticular layer, a pseudo-basement membrane and a trophoblast layer.

The birth tissue may not have been treated with an enzyme. The enzyme may be a digestive enzyme such as oxidoreductases, transferases, hydrolases, lyases, isomerases, and/or ligases, especially collagenase, protease, pepsin, or hyaluronidase. The enzyme may not be from the birth tissue. In other words, the enzyme would be exogenous to the birth tissue.

The particulates of a birth tissue, also called birth tissue particulates, may comprise viable cells. The birth tissue particulates may not comprise viable cells. The birth tissue particulates may have been cryopreserved. The birth tissue particulates may have been lyophilized or frozen. The birth tissue particulates may not have been treated with an enzyme. The enzyme may be a digestive enzyme such as oxidoreductases, transferases, hydrolases, lyases, isomerases, and/or ligases, especially collagenase, protease, pepsin, or hyaluronidase. The enzyme may not be from the birth tissue. In other words, the enzyme would be exogenous to the birth tissue.

The method may further comprise micronizing a processed birth tissue to make birth tissue particulates. The processed birth tissue may be selected from the group consisting of an umbilical cord, amniotic sac, placental plate and a combination thereof. In one embodiment, the birth tissue is an umbilical cord and the processed birth tissue is a processed umbilical cord. The processed umbilical cord may not comprise an umbilical artery. The processed umbilical cord may not comprise umbilical cord vein endothelial cells. The processed umbilical cord may comprise viable cells. The processed umbilical cord may not comprise viable cells. The processed umbilical cord may have been cryopreserved. The processed umbilical cord may have been lyophilized. In another embodiment, the birth tissue is a placental membrane and the processed birth tissue is a processed placental membrane. The processed placental membrane may have been decellularized or cryopreserved. The processed placental membrane may have been lyophilized. The processed placental membrane may comprise viable cells. The processed placenta membrane may not comprise viable cells.

The size of the birth tissue particulates or the processed birth tissue particulates mixed with a liquid to prepare an elute of a birth tissue may have an average particle size in the range of 0.1-10,000, 0.1-5,000, 0.1-2,000, 0.1-1,000, 0.1-500, 0.1-100, 0.1-10, 0.1-1, 0.5-10,000, 0.5-5,000, 0.5-2,000, 0.5-1,000, 0.5-500, 0.5-100, 0.5-10, 0.5-1, 1-10,000, 1-5,000, 1-2,000, 1-1,000, 1-500, 1-100, 1-10, 5-10,000, 5-5,000, 5-2,000, 5-1,000, 5-500, 5-100, 5-10, 10-10,000, 10-5,000, 10-2,000, 10-1,000, 10-500, 10-100, 50-10,000, 50-5,000, 50-2,000, 50-1,000, 50-500, 50-100, 100-10,000, 100-5,000, 100-2,000, 100-1,000 or 100-500 μm. For example, the birth tissue particulates or the processed birth tissue particulates may have an average particle size in the range of 10-2,000 μm.

The size of the birth tissue pieces or the processed birth tissue pieces mixed with a liquid to prepare an elute of a birth tissue may have an average surface area in the range of 0.2×0.2-50×50, 0.2×0.2-30×30, 0.2×0.2-15×15, 0.2×0.2-10×10, 0.2×0.2-5×5, 0.2×0.2-1×1, 0.2×0.2-0.5×0.5, 0.5×0.5-50×50, 0.5×0.5-30×30, 0.5×0.5-15×15, 0.5×0.5-10×10, 0.5×0.5-5×5, 0.5×0.5-1×1, 1×1-50×50, 1×1-30×30, 1×1-15×15, 1×1-10×10, 1×1-5×5, 2×2-50×50, 2×2-30×30, 2×2-15×15, 2×2-10×10, 2×2-5×5, 5×5-50×50, 5×5-30×30, 5×5-15×15, 5×5-10×10, 10×10-50×50, 10×10-30×30, 10×10-15×15, 15×15-50×50, 15×15-30×30 or 30×30-50×50 cm². For example, the birth tissue pieces or the processed birth tissue pieces may have an average surface area in the range of 1-2500 cm².

The liquid may be any liquid suitable for preserving the biological activities of the birth tissue. For example, the liquid may be a culture medium, a conditioned medium, an isotonic solution (e.g., saline and lactated Ringer's), a hypotonic solution or water. In one embodiment, the liquid is a culture medium.

The mixing step of the preparation method may be performed on a mixing device, for example, a shaker, mixer, or a rocker. The mixing step will be carried out under conditions such that the mixture is mixed at the speed of 1-5000 rpm, 1-4000 rpm, 1-3000 rpm, 1-2000 rpm, 1-1000 rpm, 1-500 rpm, 10-500 rpm, or 50-500 rpm.

The ratio of the weight of the birth tissue particulates or the processed birth tissue particulates to the volume of the liquid used in the mixing step may be in the range from 1000:1 to 1:1000, from 1000:1 to 1:500, from 1000:1 to 1:200, from 1000:1 to 1:100, from 1000:1 to 1:50, from 1000:1 to 1:10, from 1000:1 to 1:1, from 500:1 to 1:1000, from 500:1 to 1:500, from 500:1 to 1:200, from 500:1 to 1:100, from 500:1 to 1:50, from 500:1 to 1:10, from 500:1 to 1:1, from 200:1 to 1:1000, from 200:1 to 1:500, from 200:1 to 1:200, from 200:1 to 1:100, from 200:1 to 1:50, from 200:1 to 1:10, from 200:1 to 1:1, from 100:1 to 1:1000, from 100:1 to 1:500, from 100:1 to 1:200, from 100:1 to 1:100, from 100:1 to 1:50, from 100:1 to 1:10, from 100:1 to 1:1, from 50:1 to 1:1000, from 50:1 to 1:500, from 50:1 to 1:200, from 50:1 to 1:100, from 50:1 to 1:50, from 50:1 to 1:10, from 50:1 to 1:1, from 10:1 to 1:1000, from 10:1 to 1:500, from 10:1 to 1:200, from 10:1 to 1:100, from 10:1 to 1:50, from 10:1 to 1:10, from 10:1 to 1:10, from 10:1 to 1:1, from 1:1 to 1:1000, from 1:1 to 1:500, from 1:1 to 1:200, from 1:1 to 1:100, from 1:1 to 1:50, or from 1:1 to 1:10.

The ratio of the total surface area of the birth tissue pieces or the processed birth tissue pieces to the volume of the liquid used in the mixing step may be in the range from 1000:1 to 1:1000, from 1000:1 to 1:500, from 1000:1 to 1:200, from 1000:1 to 1:100, from 1000:1 to 1:50, from 1000:1 to 1:10, from 1000:1 to 1:1, from 500:1 to 1:1000, from 500:1 to 1:500, from 500:1 to 1:200, from 500:1 to 1:100, from 500:1 to 1:50, from 500:1 to 1:10, from 500:1 to 1:1, from 200:1 to 1:1000, from 200:1 to 1:500, from 200:1 to 1:200, from 200:1 to 1:100, from 200:1 to 1:50, from 200:1 to 1:10, from 200:1 to 1:1, from 100:1 to 1:1000, from 100:1 to 1:500, from 100:1 to 1:200, from 100:1 to 1:100, from 100:1 to 1:50, from 100:1 to 1:10, from 100:1 to 1:1, from 50:1 to 1:1000, from 50:1 to 1:500, from 50:1 to 1:200, from 50:1 to 1:100, from 50:1 to 1:50, from 50:1 to 1:10, from 50:1 to 1:1, from 10:1 to 1:1000, from 10:1 to 1:500, from 10:1 to 1:200, from 10:1 to 1:100, from 10:1 to 1:50, from 10:1 to 1:10, from 10:1 to 1:10, from 10:1 to 1:1, from 1:1 to 1:1000, from 1:1 to 1:500, from 1:1 to 1:200, from 1:1 to 1:100, from 1:1 to 1:50, or from 1:1 to 1:10.

The mixture may be incubated at temperature of 1-40, 1-37, 1-30, 1-25, 1-20, 1-15, 1-10, 1-4, −10-0, or −5-0° C. for a time period. The time period may be 0.5-960, 1-960, 1-840, 1-720, 1-600, 1-480, 1-360, 1-240, 1-180, 1-120, 1-60 or 1-30 hours.

The birth tissue elute may be viscous. The birth tissue elute may have a shear viscosity of 0.1-500, 1-100, 1-50, 0.1-10, 5-45 or 15-45 Pa·s at 1-5 Hz. In one embodiment, the shear viscosity of the birth tissue elute may be 5-45 Pa·s at 2.5 Hz or 0.1-10 Pa·s at 0.5 Hz. The birth tissue elute may have a shear viscosity of 0.01-0.2, 0.01-0.15, 0.01-0.1, or 0.05-0.8 Pa·s at strains higher than 10% and a shear viscosity of 0.05-10, 0.05-5, 0.05-2, 0.05-1, or 0.1-1 Pa·s at strains less than 10% at 0.5-1 Hz. The birth tissue elute may be viscous such that the birth tissue elute may not be flowable in a tube after turning the tube upside down.

The birth tissue elute may comprise double stranded DNA. The birth tissue elute may have a double strand DNA of 1-3000, 30-3000, 50-3000, 50-2000, or 100-2000 ng DNA/mL elution.

The birth tissue elute may comprise a variety of solubilized bioactive components. The solubilized bioactive components may include hyaluronic acid (HA), cytokines, growth factors, a protease inhibitor, for example, tissue inhibitor of metalloproteinase (TIMP), and/or chemokines. The preparation method may further comprise adjusting the concentration of a bioactive component in the elute to a desirable level.

The birth tissue elute may comprise a hyaluronic acid (HA). The concentration of the HA in the birth tissue elute may be 0.01-100, 0.05-50, 0.1-20, 0.5-10 mg, 1-10, or 1-5 mg/mL. The birth tissue elute may not comprise a HA that is not from the birth tissue. The preparation method may further comprise adjusting the HA concentration in the elute. The HA may contain different molecular weight, from 5 to 10,000 kDa, from 5 to 8,000 kDa, from 5 to 6,000 kDa, or from 8 to 6,000 kDa. The HA concentration may be adjusted to a desirable level at, for example, 4.5-5.5 mg/mL or 9-11 mg/mL.

The birth tissue elute may comprise one or more cytokines. The cytokine may be interleukin-1 receptor antagonist (IL-1RA), IL-4, IL-6, IL-8, IL-10, IL-11, and/or IL-13. The concentration of the IL-1RA in the birth tissue elute may be 10-2000, 50-1000, 50-500, or 10-500 ng/mL. The birth tissue elute may not comprise a cytokine that is not from the birth tissue. The preparation method may further comprise adjusting the concentration of the cytokine in the elute. The cytokine concentration may be adjusted to a desirable level at, for example, 250-350 ng/mL. The birth tissue elute may not comprise a substantial amount of IL-1. The concentration of the IL-1beta may not be higher than 10, 50, 100 or 200 pg/mL.

The birth tissue elute may comprise one or more bioactive factors (e.g., biochemical factors). The bioactive factor may be basic fibroblast growth factor (bFGF or FGF-2), transforming growth factor beta (TGF-beta), platelet derived growth factor-AA (PDGF-AA), platelet derived growth factor-BB (PDGF-BB), transforming growth factor alpha (TGF-alpha), hepatocyte growth factor (HGF), placental growth factor (PIGF), vascular endothelial growth factor (VEGF), growth differentiation factors (GDF), insulin-like growth factor (IGF), insulin-like growth factor binding protein (IGFBP), epidermal growth factor (EGF), stromal cell-derived factor-1 (SDF-1), angiogenin, pentraxin (PTX), and/or granulocyte-colony stimulating factor (GCSF). The concentration of the bFGF in the birth tissue elute may be 1-10,000 ng/mL. The birth tissue elute may not comprise a growth factor that is not from the birth tissue. The preparation method may further comprise adjusting the concentration of the growth factor in the elute. The growth factor concentration may be adjusted to a desirable level at, for example, 90-110 ng/mL.

The birth tissue elute may comprise a protease inhibitor. The birth tissue elute may comprise a protease. The birth tissue elute may not comprise a protease inhibitor that is not from the birth tissue. The birth tissue elute may not comprise a protease that is not from the birth tissue. The protease may be trypsin, serine protease, cysteine protease, threonine protease, aspartic protease, or metalloproteases. The protease inhibitor may be a tissue inhibitor of metalloproteinase (TIMP) and/or alpha-2-macroglobulin (A2M). The TIMP may be of TIMP-1, TIMP-2, TIMP-3, or TIMP-4. The concentration of the TIMP-1 in the birth tissue elute may be 0.1-100, 0.5-100, 1-100, 1-50, 1-30, 1-20 or 1-10 μg/mL. The preparation method may further comprise adjusting the concentration of the TIMP in the elute. The TIMP concentration may be adjusted to a desirable level at, for example, adjust TIMP-1 to 2.5-3.5 μg/mL or 4.5-5.5 μg/mL. The A2M concentration in the birth tissue elute may be 0.1-1000, 1-1000, 1-500, 1-100, or 10-100 μg/mL.

The birth tissue elute may comprise extracellular vesicles. The birth tissue elute may not comprise extracellular vesicles not from the birth tissue. The extracellular vesicles may be positive with a biomarker such as CD40+. The number of the extracellular vesicles in the birth tissue elute may be 10,000-100,000,000, 10,000-50,000,000, 10,000-20,000,000, 1,000,000-100,000,000, 1,000,000-50,000,000, or 1,000,000-20,000,000 per mL. The preparation method may further comprise adjusting the number of the extracellular vesicles in the elute. The extracellular vesicles may be adjusted to a desirable level at, for example, 10,000,000-50,000,000 per mL or 50,000,000-100,000,000 per mL.

The birth tissue elute may comprise exosomes. The birth tissue elute may not comprise exosomes not from the birth tissue. The exosomes may be positive with a biomarker such as CD9+. The number of the exosomes in the birth tissue elute may be 10,000-100,000,000, 10,000-50,000,000, 10,000-20,000,000, 1,000,000-100,000,000, 1,000,000-50,000,000, or 1,000,000-20,000,000 per mL. The preparation method may further comprise adjusting the number of the exosomes in the elute. The exosomes may be adjusted to a desirable level at, for example, 10,000,000-50,000,000 per mL or 50,000,000-100,000,000 per mL.

The birth tissue elute may not comprise a substantial amount (e.g., more than 90, 95, 97, 99 or 99.9 wt % or mg/ml) of solubilized extracellular matrix components. The extracellular matrix components may be selected from the group consisting of collagen, laminin, and/or fibronectin, and a combination thereof. The solubilized extracellular matrix proteins may constitute less than 0.01, 0.05, 0.1, 0.5, 1, 2, 3, 4, or 5 wt % or mg/ml of the birth tissue elute.

The birth tissue elute may not comprise a substantial amount (e.g., more than 90, 95, 97, 99 or 99.9 wt % or mg/ml) of solubilized collagen. The solubilized collagen may constitute less than 0.01, 0.05, 0.1, 0.5, 1, 2, 3, 4, or 5 wt % of the birth tissue elute. The birth tissue elute may comprise less than 0.01, 0.05, 0.1, 0.5, 1, 2, 3, 4, or 5 mg/ml collagen.

The birth tissue elute may not comprise a substantial amount (e.g., more than 90, 95, 97, 99 or 99.9 wt % or mg/ml) of solubilized laminin. The solubilized laminin may constitute less than 5, 3 or 1 wt % or mg/ml of the birth tissue elute. The birth tissue elute may comprise less than 0.01, 0.05, 0.1, 0.5, 1, 2, 3 or 5 mg/ml laminin.

A bioactive factor in the presence of the elute may have a longer shelf-life at different temperatures than the same bioactive factor in the absence of the elute. The elute may extend the shelf-life of the bioactive factor at ambient temperature from 1 minute to 48 hours. The elute may extend the shelf-life of the bioactive factor by at least 10, 100, 500 or 1,000 times. The elute may maintain from 20% to 100%, from 30% to 100%, from 30% to 80%, from 40% to 80%, or from 50% to 100% of the detectable bioactive factor at ambient temperature for 24 hours. The elute may maintain from 20% to 100%, from 30% to 100%, from 30% to 80%, from 40% to 80%, or from 50% to 100% of the detectable bioactive factor at ambient temperature for 2 days. The elute may maintain the detectable bioactive factor from 20% to 100%, from 30% to 100%, from 30% to 80%, from 40% to 80%, or from 50% to 100% at 37° C. for 24 hours. The elute may maintain from 20% to 100%, from 30% to 100%, from 30% to 80%, from 40% to 80%, or from 50% to 100% of the detectable bioactive factor at 37° C. for 2 days.

The preparation method may further comprise dehydrating, for example, by lyophilizing, also known as freeze drying, the elute. One or more agents may be added during dehydration to improve solubility of the dehydrated elute when rehydrated or re-suspended with a liquid. The preparation method may further comprise storing the elute. The elute may be stored at a temperature below 50, 40, 30, 25, 20, 15, 10, 4, or −20° C., or in the range of 1-50, 1-40, 1-30, 1-25, 1-20, 1-15, 1-10, 1-4, or 4-20° C.

For each preparation method, a birth tissue elute is provided. For example, an umbilical cord elute prepared according to any preparation method of the present invention is provided. A composition comprising a birth tissue elute, for example, an umbilical cord elute, is also provided.

A composition for treating a pathological condition in a body part of a patient in needed thereof is provided. The composition comprises an effective amount of an elute of a birth tissue and a pharmaceutically acceptable carrier. The composition may be injectable. The birth tissue may be an umbilical cord, amniotic sac, placental plate and a combination thereof. In one embodiment, the birth tissue is an umbilical cord. In another embodiment, the birth tissue is a placental membrane. The placental membrane may comprise a cellular layer, a reticular layer and a pseudo-basement membrane. The placental membrane may comprise an amniotic membrane, a cellular layer, a reticular layer and a pseudo-basement membrane. The placental membrane may comprise a cellular layer, a reticular layer, a pseudo-basement membrane and a trophoblast layer. The placental membrane may comprise an amniotic membrane, a cellular layer, a reticular layer, a pseudo-basement membrane and a trophoblast layer.

The birth tissue elute may be prepared according to the preparation method of this invention.

The composition may be viscous. The composition may be a hydrogel. The composition may have a shear viscosity of 0.05-1000, 0.05-500, 0.05-250, 0.1-500, 0.1-10, 1-100, 1-50, 5-45 or 15-45 Pa·s at 1-5 Hz. For example, the shear viscosity of the composition may be 5-45 Pa·s at 2.5 Hz or 0.1-10 Pa·s at 0.5 Hz. The birth tissue elute may have a shear viscosity of 0.05-500, 0.05-250, 0.01-0.2, 0.01-0.15, or 0.01-0.1 Pa·s at strain higher than 10% and a shear viscosity of 0.05-1000, 0.05-500, 0.05-250, 0.05-10, 0.05-5, 0.05-2, 0.05-1, or 0.1-1 Pa·s at strain less than 10% at 0.5-1 Hz.

The composition may have a freezing point from −5° C. to −80° C., from −10° C. to −80° C., from −10° C. to −60° C., from −10° C. to −50° C., from −10° C. to −40° C., or from −10° C. to −30° C.

The birth tissue elute may comprise double stranded DNA, for example, from the cells in the birth tissue. The concentration of the double strand DNA in the birth tissue elute is 1-3000, 30-3000, 50-3000, 50-2000, or 100-2000 ng/mL.

The composition may comprise a variety of bioactive components. The bioactive components may include hyaluronic acid (HA), proteoglycan, cytokines, growth factors, a protease inhibitor, for example, a tissue inhibitor of metalloproteinase (TIMP), extracellular vesicles, exosomes and/or chemokines. The concentration listed below are for the hydrated form or dehydrated composition hydrated in any type of liquid.

The composition may comprise a hyaluronic acid (HA) at, for example, 0.01-100, 0.05-50, 0.1-20, 0.5-10 mg, 1-10, or 1-5 mg/mL. The HA may contain different molecular weight, from 5 to 10,000 kDa, from 5 to 8,000 kDa, from 5 to 6,000 kDa, or from 8 to 6,000 kDa. The HA concentration may be adjusted to a desirable level at, for example, 4.5-5.5 mg/mL or 9-11 mg/mL. The HA concentration may be 4.5-5.5 mg/mL or 9-11 mg/mL. The HA concentration in the composition may be at least of 0.3, 0.5, 0.6, 0.7, 0.8, 0.9 or 1 mg/mL.

The composition may comprise one or more cytokines. The cytokine may be interleukin-1 receptor antagonist (IL-1RA), IL-4, IL-6, IL-10, IL-11, and/or IL-13. The concentration of the IL-1RA in the composition may be 10-2000, 50-1000, 50-500, or 10-500 ng/mL. The IL-1RA concentration may be adjusted to a desirable level at, for example, 250-350 ng/mL. The composition may not comprise a substantial amount of IL-1. The concentration of the IL-1beta may not be higher than 10, 50, 100 or 200 μg/mL.

The composition may comprise one or more bioactive factors. The bioactive factor may be basic fibroblast growth factor (bFGF or FGF-2), transforming growth factor beta (TGF-beta), platelet derived growth factor-AA (PDGF-AA), platelet derived growth factor-BB (PDGF-BB), transforming growth factor alpha (TGF-alpha), hepatocyte growth factor (HGF), placental growth factor (PIGF), vascular endothelial growth factor (VEGF), growth differentiation factors (GDF), insulin-like growth factor (IGF), insulin-like growth factor binding protein (IGFBP), epidermal growth factor (EGF), angiogenin, pentraxin (PTX), stromal cell-derived factor-1 (SDF-1), and/or granulocyte-colony stimulating factor (GCSF). The concentration of the TGF-beta3 in the composition may be 1-100, 1-50, 2-40, 2-30, or 2-20 ng/mL. The TGF-beta3 concentration in the composition may be at least of 0.5, 1, 2, 2.5, 3, 3.5, 4, 4.5, or 5 ng/mL. The PTX-3 concentration may be 1-500, 10-500, 20-400, 20-300, or 20-200 ng/mL. The PTX-3 concentration in the composition may be at least of 10, 20, 30, 40, or 50 ng/mL. The HGF concentration may be 0.1-100, 0.1-80, 0.1-50, 0.1-30, 0.1-20, 0.1-10, or 0.2-20 ng/mL. The HGF concentration in the composition may be at least of 0.1, 0.2, 0.3, 0.4, or 0.5 ng/mL.

The composition may comprise a protease inhibitor. The protease inhibitor may be a tissue inhibitor of metalloproteinase (TIMP) and alpha-2 macroglobulin (A2M). The TIMP may be of TIMP-1, TIMP-2, TIMP-3, or TIMP-4. The concentration of the TIMP-1 in the composition may be 1-10,000, 1-1000, 10-1000, 10-500, 40-400, 50-500 or 40-300 ng/mL. The TIMP1 concentration in the composition may be at least of 10, 30, 60, 80, 100, 200, 300, 400, 500, 600, 800, or 1000 ng/mL. The TIMP2 concentration may be 1-1000, 5-1000, 10-500, 10-400, 20-400, 20-200, 20-300, 30-200, or 30-500 ng/mL. The TIMP2 concentration in the composition may be at least of 20, 30, 40, 50, 60, 70, 80, 90, 100, 200, or 300 ng/mL. The TIMP3 concentration may be 0.1-100, 0.2-50, 0.5-50, 0.5-40, 1-100, 1-50, 1-30, or 0.5-10 ng/mL. The TIMP3 concentration in the composition may be at least of 0.2, 0.3, 0.4, 0.5, 0.6, 0.7, 0.8, 0.9, or 1 ng/mL. The A2M concentration may be 1-1000, 1-800, 3-500, 5-500, 3-300, 3-200, or 3-100 μg/mL. The A2M concentration in the composition may be at least of 1, 2, 3, 4, 5, 6, 7, 8, 9, or 10 μg/mL.

The composition may comprise extracellular vesicles. The extracellular vesicles may be CD40+. The number of the extracellular vesicles in the composition may be 10,000-100,000,000, 10,000-50,000,000, 10,000-20,000,000, 1,000,000-100,000,000, 1,000,000-50,000,000, or 1,000,000-20,000,000. The preparation method may further comprise adjusting the number of the extracellular vesicles in the composition. The extracellular vesicles may be adjusted to a desirable level at, for example, 10,000,000-50,000,000 per mL or 50,000,000-100,000,000 per mL.

The composition may comprise exosomes. The exosomes may be CD9+. The number of the exosomes in the composition may be 10,000-100,000,000, 10,000-50,000,000, 10,000-20,000,000, 1,000,000-100,000,000, 1,000,000-50,000,000, or 1,000,000-20,000,000 per mL. The preparation method may further comprise adjusting the number of the exosomes in the composition. The exosomes may be adjusted to a desirable level at, for example, 10,000,000-50,000,000 per mL or 50,000,000-100,000,000 per mL.

The composition may not comprise a substantial amount (e.g., more than 90, 95, 97, 99 or 99.9 wt % or mg/ml) of solubilized extracellular matrix components. The extracellular matrix components may be selected from the group consisting of collagen, laminin, proteoglycan, glycosaminoglycan, lipid, and/or fibronectin, and a combination thereof. The solubilized proteoglycan 4 (lubricin) may be 0.1-500, 0.5-400, 0.5-300, 0.5-200, 1-200, 1-100, or 10-100 ng/mL. The solubilized extracellular matrix components may constitute less than 0.01, 0.05, 0.1, 0.5, 1, 2, 3, 4, or 5 wt % or mg/ml of the composition. The solubilized collagen may constitute less than 0.01, 0.05, 0.1, 0.5, 1, 2, 3, 4, or 5 wt % or mg/ml of the composition. The solubilized laminin may constitute less than 0.01, 0.05, 0.1, 0.5, 1, 2, 3, 4, or 5 wt % or mg/ml of the birth tissue elute. The composition may comprise viable cells. The composition may not comprise viable cells. The composition may not comprise a viable cell.

The composition may have been cryopreserved. The composition may have been frozen below the freezing point of the water. The composition may be lyophilized.

A bioactive factor in the presence of the elute may have a longer shelf-life at different temperatures than the same bioactive factor in the absence of the elute. The elute may extend the shelf-life of the bioactive factor at ambient temperature from 1 minute to 48 hours. The elute may extend the shelf-life of the bioactive factor by at least 10, 100, 500 or 1,000 times. The elute may maintain from 20% to 100%, from 30% to 100%, from 30% to 80%, from 40% to 80%, or from 50% to 100% of the detectable bioactive factor at ambient temperature for 24 hours. The elute may maintain from 20% to 100%, from 30% to 100%, from 30% to 80%, from 40% to 80%, or from 50% to 100% of the detectable bioactive factor at ambient temperature for 2 days. The elute may maintain the detectable bioactive factor from 20% to 100%, from 30% to 100%, from 30% to 80%, from 40% to 80%, or from 50% to 100% at 37° C. for 24 hours. The elute may maintain from 20% to 100%, from 30% to 100%, from 30% to 80%, from 40% to 80%, or from 50% to 100% of the detectable bioactive factor at 37° C. for 2 days.

The composition may further comprise umbilical cord particulates. The umbilical cord particulates may have an average particle size in the range of 0.1-10,000, 0.1-5,000, 0.1-2,000, 0.1-1,000, 0.1-500, 0.1-100, 0.1-10, 0.1-1, 0.5-10,000, 0.5-5,000, 0.5-2,000, 0.5-1,000, 0.5-500, 0.5-100, 0.5-10, 0.5-1, 1-10,000, 1-5,000, 1-2,000, 1-1,000, 1-500, 1-100, 1-10, 5-10,000, 5-5,000, 5-2,000, 5-1,000, 5-500, 5-100, 5-10, 10-10,000, 10-5,000, 10-2,000, 10-1,000, 10-500, 10-100, 50-10,000, 50-5,000, 50-2,000, 50-1,000, 50-500, 50-100, 100-10,000, 100-5,000, 100-2,000, 100-1,000 or 100-500 μm. For example, the umbilical cord particulates may have an average particle size in the range of 10-2,000 μm. The umbilical cord particulates may comprise viable cells. The umbilical cord particulates may not comprise viable cells. The umbilical cord particulates may have been cryopreserved. The umbilical cord particulates may have been frozen. The umbilical cord particulates may have been lyophilized. The umbilical cord particulates may be have been decellularized. Alternatively, the umbilical cord particulates may not have been decellularized.

The composition may further comprise particulates of a placenta membrane, also referred to as placenta membrane particulates. The placental membrane may comprise a cellular layer, a reticular layer and a pseudo-basement membrane. The placental membrane may comprise an amniotic membrane, a cellular layer, a reticular layer and a pseudo-basement membrane. The placental membrane may comprise a cellular layer, a reticular layer, a pseudo-basement membrane and a trophoblast layer. The placental membrane may comprise an amniotic membrane, a cellular layer, a reticular layer, a pseudo-basement membrane and a trophoblast layer. The placenta membrane particulates may have an average particle size in the range of 0.1-10,000, 0.1-5,000, 0.1-2,000, 0.1-1,000, 0.1-500, 0.1-100, 0.1-10, 0.1-1, 0.5-10,000, 0.5-5,000, 0.5-2,000, 0.5-1,000, 0.5-500, 0.5-100, 0.5-10, 0.5-1, 1-10,000, 1-5,000, 1-2,000, 1-1,000, 1-500, 1-100, 1-10, 5-10,000, 5-5,000, 5-2,000, 5-1,000, 5-500, 5-100, 5-10, 10-10,000, 10-5,000, 10-2,000, 10-1,000, 10-500, 10-100, 50-10,000, 50-5,000, 50-2,000, 50-1,000, 50-500, 50-100, 100-10,000, 100-5,000, 100-2,000, 100-1,000 or 100-500 μm. For example, the placenta membrane particulates may have an average particle size in the range of 10-2,000 μm. The placenta membrane particulates may comprise viable cells. The placenta membrane particulates may not comprise viable cells. The placenta membrane particulates may have been cryopreserved. The placenta membrane particulates may have been frozen. The placenta membrane particulates may have been lyophilized. The placenta membrane particulates may have been decellularized. Alternatively, the placenta membrane particulates may not have been decellularized. The placenta membrane particulates may have been denatured.

The composition may further comprise particulates of placental membrane and particulates of umbilical cord and the ratio of placental membrane particulates and umbilical cord particulates may be 10:1, 8:1, 6:1, 5:1, 4:1, 3:1, 2:1, 1:1, 1:2, 1:3, 1:4, 1:5, 1:6, 1:8, or 1:10 in wet weight or dry weight. The placental membrane particulates and/or umbilical cord particulates (solid portion) may be covered by the bioactive factors in the elute (soluble portion) in the lyophilized form and/or hydrated form.

Various compositions comprising an elute of a first birth tissue and particulates of a second birth tissue may be prepared. The first and second birth tissue may be the same or different. Each of the first and second birth tissue may consist of one or more birth tissue types. Examples of the birth tissue types include an umbilical cord, an amniotic sac, a placental plate, or a combination thereof.

The elute is prepared from the first birth tissue. Particulates of a first birth tissue may be mixed with a liquid to form a mixture, which is then incubated before a supernatant is collected from the mixture. The first birth tissue may be an umbilical cord, an amniotic sac, a placental plate or a combination thereof. The placental membrane may comprise amniotic membrane, chorionic membrane, trophoblast layer or a combination thereof. These membrane layers may be separated or non-separated, preferably non-separated. The ratio of the weight of the first birth tissue particulates to the volume of the liquid may be in the range from 1:1 to 1:100. The incubation may be carried out at a temperature of, for example, from −5° C. to 15° C. for 1-240 hours.

The particulates are prepared from the second birth tissue. The second birth tissue may be an umbilical cord, an amniotic sac, a placental plate or a combination thereof. The placental membrane may comprise amniotic membrane, chorionic membrane, trophoblast layer or a combination thereof. These membrane layers may be separated or non-separated, preferably non-separated. The second birth tissue may be decellularized or non-decellularized. For example, the particulates may be prepared from a non-decellularized umbilical cord tissue or a decellularized placental membrane, which includes amniotic membrane, chorionic membrane and trophoblast layer.

The elute of the first birth tissue and the particulates of the second birth tissue may be mixed to prepare a composition. In the composition, the elute and the particulates may have a ratio of an elute volume (milliliter) to particulates dry weight (gram) in the range from 1000:1 to 1:1, from 500:1 to 1:1, from 100:1 to 1:1, from 80:1 to 1:1, from 40:1 to 1:1, from 30:1 to 1:1, from 20:1 to 1:1, from 10:1 to 1:1, from 5:1 to 1:1, from 4:1 to 1:1, from 3:1 to 1:1, or from 2:1 to 1:1. In the composition, the elute and the particulates may have a ratio of an elute volume (milliliter) to particulates wet weight (gram) from 100:1 to 1:10, from 50:1 to 1:10, from 20:1 to 1:10, from 10:1 to 1:10, from 5:1 to 1:10, from 4:1 to 1:10, from 3:1 to 1:10, from 2:1 to 1:10, from 1:1 to 1:10, from 1:2 to 1:10, from 1:3 to 1:10, or from 1:5 to 1:10. The composition comprising the dry particulates in the elute may be in a concentration of 1-80%, 1-60%, 1-50%, 1-40%, 1-30%, 1-20%, or 1-10% (gram per 100 milliliter).

The composition may comprise various combinations of the elute of the first birth tissue and the particulates of the second birth tissue. The elute may be prepared from one or more birth tissues of non-decellularized or decellularized 1) an umbilical cord, 2) placental plate, or 3) placental membrane including amniotic membrane, chorionic membrane and trophoblast layer, while the particulates may be from one or more non-decellularized or decellularized 1) umbilical cord, 2) placental plate, 3) placental membrane, including amniotic membrane, chorionic membrane and trophoblast layer. Exemplary compositions include:

(1) an elute of an umbilical cord and particulates of non-decellularized umbilical cord;

(2) an elute of an umbilical cord and particulates of decellularized umbilical cord;

(3) an elute of an umbilical cord and particulates of non-decellularized placental membrane, including amniotic membrane, chorionic membrane and trophoblast layer, which are non-separated;

(4) an elute of an umbilical cord and particulates of decellularized placental membrane, including amniotic membrane, chorionic membrane and trophoblast layer, which are non-separated;

(5) an elute of an umbilical cord and particulates of non-decellularized umbilical cord and non-decellularized placental membrane, including amniotic membrane, chorionic membrane and trophoblast layer, which are non-separated;

(6) an elute of an umbilical cord and particulates of non-decellularized umbilical cord and decellularized placental membrane, including amniotic membrane, chorionic membrane and trophoblast layer, which are non-separated;

(7) an elute of an umbilical cord and particulates of decellularized umbilical cord and non-decellularized placental membrane, including amniotic membrane, chorionic membrane and trophoblast layer, which are non-separated;

(8) an elute of an umbilical cord and particulates of decellularized umbilical cord and decellularized placental membrane, including amniotic membrane, chorionic membrane and trophoblast layer, which are non-separated;

(9) an elute of a placental membrane, including amniotic membrane, chorionic membrane and trophoblast layer, which are non-separated, and particulates of non-decellularized umbilical cord;

(10) an elute of a placental membrane, including amniotic membrane, chorionic membrane and trophoblast layer, which are non-separated, and particulates of decellularized umbilical cord;

(11) an elute of a placental membrane, including amniotic membrane, chorionic membrane and trophoblast layer, which are non-separated, and particulates of non-decellularized placental plate, including amniotic membrane, chorionic membrane and trophoblast layer, which are non-separated;

(12) an elute of a placental membrane, including amniotic membrane, chorionic membrane and trophoblast layer, which are non-separated, and particulates of decellularized placental plate, including amniotic membrane, chorionic membrane and trophoblast layer, which are non-separated;

(13) an elute of a placental membrane, including amniotic membrane, chorionic membrane and trophoblast layer, which are non-separated, and particulates of non-decellularized umbilical cord and non-decellularized placental plate, including amniotic membrane, chorionic membrane and trophoblast layer, which are non-separated;

(14) an elute of a placental membrane, including amniotic membrane, chorionic membrane and trophoblast layer, which are non-separated, and particulates of non-decellularized umbilical cord and decellularized placental plate, including amniotic membrane, chorionic membrane and trophoblast layer, which are non-separated;

(15) an elute of a placental membrane, including amniotic membrane, chorionic membrane and trophoblast layer, which are non-separated, and particulates of decellularized umbilical cord and non-decellularized placental plate, including amniotic membrane, chorionic membrane and trophoblast layer, which are non-separated; or

(16) an elute of a placental membrane, including amniotic membrane, chorionic membrane and trophoblast layer, which are non-separated, and particulates of decellularized umbilical cord and decellularized placental plate, including amniotic membrane, chorionic membrane and trophoblast layer, which are non-separated;

The composition may be aliquoted and packaged. The composition may be freeze-dried, cryopreserved, or frozen. The composition may be sterilized by, for example, gamma irradiation, e-beam, Ethylene Oxide (EO), or critical CO2.

A composition comprising an elute and particulates of placental membrane and particulates of umbilical cord may be viscous. The composition may be a hydrogel. The composition may have a shear viscosity of 0.05-1000, 0.05-500, 0.05-250, 0.1-500, 1-100, 1-50, 5-45 or 15-45 Pa·s at 1-5 Hz. For example, the shear viscosity of the composition may be 5-45 Pa·s at 2.5 Hz or 0.1-500 Pa·s at 0.5 Hz. The birth tissue elute may have a shear viscosity of 0.05-500, 0.05-250, 0.01-0.2, 0.01-0.15, or 0.01-0.1 Pa·s at strain higher than 10% and a shear viscosity of 0.05-1000, 0.05-500, 0.05-250, 0.05-10, 0.05-5, 0.05-2, 0.05-1, or 0.1-1 Pa·s at strain less than 10% at 0.5-1 Hz.

The composition may have a freezing point from −5° C. to −80° C., from −10° C. to −80° C., from −10° C. to −60° C., from −10° C. to −50° C., from −10° C. to −40° C., or from −10° C. to −30° C.

The composition may comprise double strand DNA of 1-10,000, 50-5000, 20-2000, 10-1000 ng/mg dry tissue weight, 1-1000, 1-500, 20-200, 10-100 ng/mg wet tissue weight.

The composition may comprise a variety of bioactive components. The bioactive components may include hyaluronic acid (HA), proteoglycan, cytokines, growth factors, a protease inhibitor, for example, a tissue inhibitor of metalloproteinase (TIMP), extracellular vesicles, exosomes and/or chemokines. The concentration listed below are for the hydrated form or dehydrated composition hydrated in any type of liquid.

The composition may comprise a hyaluronic acid (HA) at, for example, 0.01-100, 0.05-50, 0.1-20, 0.5-10 mg, 1-10, or 1-5 mg/mL. The HA may contain different molecular weight, from 5 to 10,000 kDa, from 5 to 8,000 kDa, from 5 to 6,000 kDa, or from 8 to 6,000 kDa. The HA concentration may be adjusted to a desirable level at, for example, 4.5-5.5 mg/mL or 9-11 mg/mL. The HA concentration may be 4.5-5.5 mg/mL or 9-11 mg/mL. The HA concentration in the composition may be at least of 0.5, 1, 1.5, 2, 2.5, or 3 mg/mL.

The composition may comprise one or more cytokines. The cytokine may be interleukin-1 receptor antagonist (IL-1RA), IL-4, IL-6, IL-10, IL-11, and/or IL-13. The concentration of the IL-1RA in the composition may be 10-2000, 50-1000, 50-500, or 10-500 ng/mL. The cytokine concentration may be 250-350 ng/mL. The composition may not comprise a substantial amount of IL-1. The concentration of the IL-1beta may not be higher than 10 pg/mg dry tissue weight. The concentration of the IL-1beta may not be higher than 10, 50, 100 or 200 pg/mL.

The composition may comprise one or more bioactive factors. The bioactive factor may be basic fibroblast growth factor (bFGF or FGF-2), transforming growth factor beta (TGF-beta), platelet derived growth factor-AA (PDGF-AA), platelet derived growth factor-BB (PDGF-BB), transforming growth factor alpha (TGF-alpha), hepatocyte growth factor (HGF), placental growth factor (PIGF), vascular endothelial growth factor (VEGF), growth differentiation factors (GDF), insulin-like growth factor (IGF), insulin-like growth factor binding protein (IGFBP), epidermal growth factor (EGF), angiogenin, pentraxin (PTX), stromal cell-derived factor-1 (SDF-1), and/or granulocyte-colony stimulating factor (GCSF). The concentration of the TGF-beta3 in the composition may be 1-100, 1-50, 2-40, 2-30, or 2-20 ng/mL. The TGF-beta3 concentration in the composition may be at least of 0.5, 1, 2, 2.5, 3, 3.5, 4, 4.5, or 5 ng/mL. The PTX-3 concentration may be 1-500, 10-500, 20-400, 20-300, or 20-200 ng/mL. The PTX-3 concentration in the composition may be at least of 10, 20, 30, 40, or 50 ng/mL. The HGF concentration may be 1-1000, 1-800, 1-500, 1-300, 10-200, 10-400, or 20-400 ng/mL. The HGF concentration in the composition may be at least of 1, 2, 3, 4, or 5 ng/mL.

The composition may comprise a protease inhibitor. The protease inhibitor may be a tissue inhibitor of metalloproteinase (TIMP) and alpha-2 macroglobulin (A2M). The TIMP may be of TIMP-1, TIMP-2, TIMP-3, or TIMP-4. The concentration of the TIMP-1 in the composition may be 1-10,000, 1-1000, 10-1000, 10-500, 40-400, 50-500 or 40-300 ng/mL. The TIMP1 concentration in the composition may be at least of 10, 30, 60, 80, 100, 200, 300, 400, 500, 600, 800, or 1000 ng/mL. The TIMP2 concentration may be 1-10,000, 5-10,000, 10-5000, 10-4000, 20-4000, 20-2000, 30-3000, 40-2000, 40-1000, or 40-1000 ng/mL. The TIMP2 concentration in the composition may be at least of 20, 30, 40, 50, 60, 70, 80, 90, 100, 200, 300, 400, 500, 600, 700, 800, or 1000 ng/mL. The TIMP3 concentration may be 0.1-100, 0.5-100, 0.5-50, 0.5-40, 1-100, 1-50, 1-30 or 1-10 ng/mL. The TIMP3 concentration in the composition may be at least of 0.5, 1, 2, 3, 4, 5, 6, 8, 9, or 10 ng/mL. The A2M concentration may be 1-1000, 1-800, 3-500, 5-500, 3-300, 3-200, or 3-100 μg/mL. The A2M concentration in the composition may be at least of 1, 2, 3, 4, 5, 6, 7, 8, 9, or 10 μg/mL.

The composition may not comprise a substantial amount (e.g., more than 90, 95, 97, 99 or 99.9 wt % or mg/ml) of soluble extracellular matrix components. The extracellular matrix components may be selected from the group consisting of collagen, laminin, proteoglycan, glycosaminoglycan, lipid, and/or fibronectin, and a combination thereof. The solubilized proteoglycan 4 (lubricin) may be 0.1-500, 0.5-400, 0.5-300, 0.5-200, 1-200, 1-100, or 10-100 ng/mL. The solubilized extracellular matrix components may constitute less than 0.01, 0.05, 0.1, 0.5, 1, 2, 3, 4, or 5 wt % or mg/ml of the composition. The solubilized collagen may constitute less than 0.01, 0.05, 0.1, 0.5, 1, 2, 3, 4, or 5 wt % or mg/ml of the composition. The solubilized laminin may constitute less than 0.01, 0.05, 0.1, 0.5, 1, 2, 3, 4, or 5 wt % or mg/ml of the birth tissue elute. The composition may comprise viable cells. The composition may not comprise viable cells.

The composition may have been cryopreserved. The composition may have been frozen below the freezing point of the water. The composition may be lyophilized.

A bioactive factor in the presence of the elute composition may have a longer shelf-life at different temperatures than the same bioactive factor in the absence of the elute. The elute may extend the shelf-life of the bioactive factor at ambient temperature from 1 minute to 48 hours. The elute may extend the shelf-life of the bioactive factor by at least 10, 100, 500 or 1,000 times. The elute may maintain from 20% to 100%, from 30% to 100%, from 30% to 80%, from 40% to 80%, or from 50% to 100% of the detectable bioactive factor at ambient temperature for 24 hours. The elute may maintain from 20% to 100%, from 30% to 100%, from 30% to 80%, from 40% to 80%, or from 50% to 100% of the detectable bioactive factor at ambient temperature for 2 days. The elute may maintain the detectable bioactive factor from 20% to 100%, from 30% to 100%, from 30% to 80%, from 40% to 80%, or from 50% to 100% at 37° C. for 24 hours. The elute may maintain from 20% to 100%, from 30% to 100%, from 30% to 80%, from 40% to 80%, or from 50% to 100% of the detectable bioactive factor at 37° C. for 2 days.

The function of bioactive factor in the composition with the elute and placental membrane particulates and/or umbilical cord particulates may be 10-200%, 10-150%, 10-100%, or 50-100% higher than the composition of placental membrane particulates and/or umbilical cord particulates without elute after 5 minutes application. The function of bioactive factor in the composition with the elute may be 10-200%, 10-150%, 10-100%, or 50-100% higher than the composition without elute after 15 minutes application. The function of bioactive factor in the composition with the elute may be 10-200%, 10-150%, 10-100%, or 50-100% higher than the composition without elute after 30 minutes application. The function of bioactive factor in the composition with the elute may be 10-200%, 10-150%, 10-100%, or 50-100% higher than the composition without elute after 60 minutes application.

Without any enzymatic treatment, for example no protease digestion, the detectable quantity of bioactive factor in the composition with an elute and particulates may be 10-200%, 10-150%, 10-100%, or 50-100% higher than the composition of particulates without an elute after 1 minute of hydration, after 5 minutes hydration, or after 10 minutes of hydration. The detectable concentration of bioactive factor in an application site may be 10-200%, 10-150%, 10-100% or 50-100% higher for the composition with an elute than the composition without an elute after 1 minute, 5 minutes, or 15 minutes application.

With respect to the composition of the present invention, the body part may be a joint or tissue. The joint may be selected from the group consisting of knee, shoulder, hip, elbow, wrist, fingers, toes, and ankle joints. The join may be a knee joint. The tissue may be selected from the group consisting of tendon, ligament, bursa, fascia, cartilage, muscle, connective tissue, dermis, synovium, and enthesis. The tissue may be selected from the group consisting of osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain and muscle pain. The pathological condition may be osteoarthritis, bursitis or fasciitis. In one embodiment, the pathological condition is inflammation.

The composition may be in vivo sustainable. The composition may remain at least 10, 20, 30, 40, 50, 60, 70 or 80% effective for a time period. The time period may be at least 0.5, 1, 2, 3, 4, 5 or 6 months or 1-2, 1-3 or 1-6 months.

A method for treating a pathological condition in a body part of a patient in need thereof is provided. The treatment method comprises administering to the body part of the patient an effective amount of the composition of the present invention or a placental membrane sheet. The composition may be injected into the body part.

According to the treatment method, the placental membrane sheet may comprise a cellular layer, a reticular layer and a pseudo-basement membrane. The placental membrane sheet may comprise an amniotic membrane, a cellular layer, a reticular layer and a pseudo-basement membrane. The placental membrane sheet may comprise a cellular layer, a reticular layer, a pseudo-basement membrane and a trophoblast layer. The placental membrane sheet may comprise an amniotic membrane, a cellular layer, a reticular layer, a pseudo-basement membrane and a trophoblast layer. The placental membrane sheet may have a thickness of 50-800 μm. The placental membrane sheet has fenestration. The placental membrane sheet may have liquid absorption of 90-99%. The placenta membrane sheet may have been decellularized. The placental membrane sheet may have a DNA content at least 90% less than that of a control non-decellularized placental membrane. A control non-decellularized placental membrane have the same structure as the placental membrane in the placental membrane sheet except that the placental membrane in the placental membrane sheet may have been decellularized while the control non-decellularized placental membrane has not been decellularized. The placenta membrane sheet may not have been denatured.

According to the treatment method, the body part may not be on the surface of the patient. The body part may be a joint or tissue. The joint may be selected from the group consisting of knee, shoulder, hip, elbow, wrist, finger, toe and ankle joints. The join may be a knee joint. The tissue may be selected from the group consisting of tendon, ligament, bursa, fascia, cartilage, muscle, connective tissue, dermis, synovium, and enthesis. The tissue is a soft tissue surrounding a joint.

According to the treatment method, the pathological condition may be selected from the group consisting of osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain, arthroplasty, and muscle pain. The pathological condition may be selected from the group consisting of osteoarthritis, bursitis and fasciitis. The pathological condition may be inflammation. The pathological condition may be a degenerative tissue defect. In one embodiment, prior to injection into a patient, the composition of the present invention can be mixed with PRP or cells prepared from the patient, or from a donor. In another embodiment, prior to applying placental membrane sheet into a patient, the placental membrane sheet of the present invention can be hydrated with PRP or cells prepared from the patient, or from a donor.

Where the body part has a cutaneous wound, the treatment method may further comprise applying the placental membrane sheet onto the wound. The treatment method may further comprise applying a porous soft tissue scaffold or a porous sponge-like structure onto the wound after the placental membrane sheet is applied onto the wound.

Where the pathological condition is osteoarthritis or synovitis in a joint and the joint comprises an inflamed synovial tissue, the treatment method may further comprise injecting the composition of the present invention into the inflamed synovial tissue. The patient may have received an open join surgery. The patient may have received an arthroscopic joint surgery. In one embodiment, the patient may receive an injection of the current invention into the wound site of joint after the inflamed synovial tissue is removed.

Where the pathological condition is osteoarthritis or synovitis in a joint and the joint has wound after an inflamed synovial tissue is removed from the joint, the treatment method may further comprise applying the placental membrane sheet onto the wound by, for example, suturing, gluing, or stapling. The patient may have received an open join surgery. The patient may have received an arthroscopic joint surgery.

The treatment method may further comprise reducing adhesiveness of the body part. An effective amount of the composition of the present invention or a placental membrane sheet may be applied to the body part. The adhesiveness of the body part may be reduced by at least 5%, 10%, 15%, 20%, 25%, 30%, 35%, 40%, 45% or 50%.

The treatment method may further comprise improving healing of the body part. The healing may be tendon-to-bone healing. An effective amount of the composition of the present invention or a placental membrane sheet may be applied to the body part. The healing of the body part may be improved by at least 5%, 10%, 15%, 20%, 25%, 30%, 35%, 40%, 45% or 50%.

The treatment method may further comprise improving incorporation of an implant into the body part. An effective amount of the composition of the present invention or a placental membrane sheet may be applied to implant and/or the body part. The implant may be selected from the group consisting of allograft, xenograft, silicone implant, metal implant, device implant, breast implant, pacemaker implant, microchip implant, drug delivery device implant, and internal monitor implant. The implant may comprise a placental membrane. The placental membrane may comprise a cellular layer, a reticular layer and a pseudo-basement membrane. The placental membrane may comprise an amniotic membrane, a cellular layer, a reticular layer and a pseudo-basement membrane. The placental membrane may comprise a cellular layer, a reticular layer, a pseudo-basement membrane and a trophoblast layer. The placental membrane may comprise an amniotic membrane, a cellular layer, a reticular layer, a pseudo-basement membrane and a trophoblast layer. The placenta membrane may have been decellularized. The placenta membrane may not have been denatured. The incorporation of the implant into the body part may be improved by at least 5%, 10%, 15%, 20%, 25%, 30%, 35%, 40%, 45% or 50%.

The treatment method may further comprise wrapping a tissue with a placental membrane sheet. The tissue may be selected from the group consisting of a nerve, a tendon, a ligament, a bone, a muscle and a combination thereof.

The treatment method may further comprise recellularization of the placental membrane sheet in the patient with cells. The cells may be recipient cells. The cells may be selected from the group consisting of fibroblasts, endothelial cells, stem cells, keratinocytes, macrophages, synoviocytes, chondrocytes, tenocytes, myoblasts, myocytes, progenitor cells, and epithelial cells. For example, the cells may be synoviocytes, fibroblasts or a combination thereof.

The treatment method may further comprise growing cells in the placental membrane sheet. The cells may be recipient cells. The cells may be selected from the group consisting of fibroblasts, endothelial cells, stem cells, keratinocytes, macrophages, synoviocytes, chondrocytes, tenocytes, myoblasts, myocytes, progenitor cells, and epithelial cells. For example, the cells may be synoviocytes, fibroblasts or a combination thereof.

The treatment method may further comprise migrating cells in the placental membrane sheet. The cells may be recipient cells. The cells may be selected from the group consisting of fibroblasts, endothelial cells, stem cells, keratinocytes, macrophages, synoviocytes, chondrocytes, tenocytes, myoblasts, myocytes, progenitor cells, and epithelial cells. For example, the cells may be synoviocytes, fibroblasts or a combination thereof.

The treatment method may further comprise remodeling the placental membrane sheet by cells. The cells may be recipient cells. The cells may be selected from the group consisting of fibroblasts, endothelial cells, stem cells, keratinocytes, macrophages, synoviocytes, chondrocytes, tenocytes, myoblasts, myocytes, progenitor cells, and epithelial cells. For example, the cells may be synoviocytes, fibroblasts or a combination thereof.

### Example 1. Dissection and Preparation of Umbilical Cord

Human term placenta and amniotic sac with research consent were obtained after caesarean section and transferred to the processing facility under sterile condition.

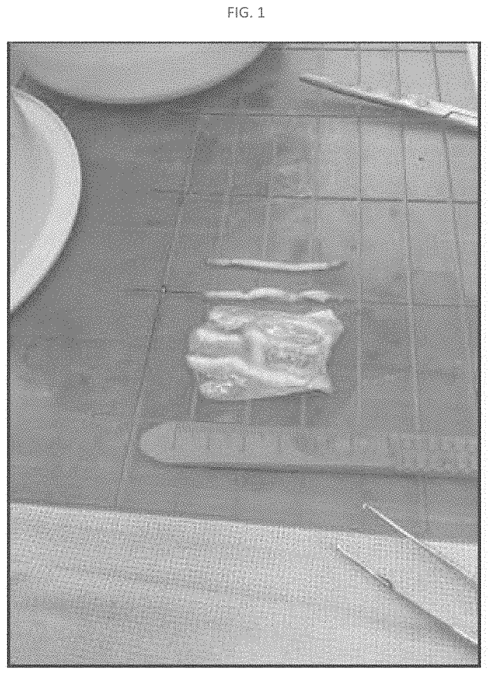

The umbilical cord attached to the placenta was cut at the cord-placenta junction and rinsed with ice-cold saline. Umbilical cord was further cut into segments (5-6 cm long) in the saline and loosely bond blood clots along the umbilical cords were removed. Umbilical cord segments were transferred to a plastic petri dish for dissection. A longitudinal cut was made by a scalpel along the length of the umbilical cord segment to expose the umbilical cord arteries. The arteries were then dissected out from the surrounding tissues with forceps and scissors (FIG. 1). The umbilical cord vein was cut open to expose the luminal side and the endothelial cells were scraped off the umbilical cord vein with a blade. The processed umbilical cords were transferred to an ice-cold DMEM medium until further process.

### Example 2. Cryopreservation of Umbilical Cord

The umbilical cord segments as processed in Example 1 were transferred from the DMEM medium to 50 mL conical tubes. A sufficient amount of a cryopreservation medium was added to the tubes such that umbilical cord segments were completely submerged.

Some umbilical cord segments from Example 1 were cut to small pieces (˜0.5 cm), transferred to 50 mL conical tubes and stored at −80° C. For cryopreservation of small pieces of umbilical cord segments, sufficient amount of a cryopreservation medium was added to the tubes such that umbilical cord pieces were completely submerged.

The tubes were then placed in a Styrofoam box and transferred to a −80° C. freezer for freezing and short-term storage. After at least 24 hours in the −80° C. freezer, some cryopreserved umbilical cords in the tubes were transferred to liquid nitrogen for long-term storage.

### Example 3. Preparation of Umbilical Cord Particulates without Cryopreservation

The umbilical cords in small pieces (˜0.5 cm) made from example 2 were used for micronization. A cryomill (Retsch) or a grinder was used to micronize the small frozen umbilical cords pieces. The frozen umbilical cord pieces were transferred to a grinding jar with a grinding ball, and then the jar was sealed. The grinding jar was pre-cooled by liquid nitrogen prior to the grinding process. The frozen umbilical cord pieces were pulverized for 15 mins at 30 Hz with the grinding jar being continually cooled with liquid nitrogen. As a result, the small umbilical cord pieces were micronized into umbilical cord particulates.

The umbilical cord particulates were transferred to sterile 50 mL conical tubes from the grinding jar and the weight of the umbilical cord particulates was recorded. The umbilical cord particulates were then aliquoted and stored at −80° C. or lyophilized.

### Example 4. Preparation of Cryopreserved Umbilical Cord Particulates

The cryopreserved umbilical cords in small pieces (˜0.5 cm) made from example 2 will be used for micronization. A cryomill (Retsch) or a grinder is used to micronize the small frozen, cryopreserved umbilical cords pieces. The frozen umbilical cord pieces are transferred to a grinding jar with a grinding ball, and then the jar was sealed. The grinding jar is pre-cooled by liquid nitrogen prior to the grinding process. The frozen umbilical cord pieces are pulverized for 15 mins at 30 Hz with the grinding jar being continually cooled with liquid nitrogen. As a result, the umbilical cords are micronized into cryopreserved umbilical cord particulates.

### Example 5. Preparation of an Umbilical Cord Conditioned Medium

A DMEM medium with 1% antibiotics was added to the umbilical cord particulates at 2 mL medium per gram of the umbilical cord particulates in 50 mL conical tubes to form a medium-umbilical cord particulate suspension, which was mixed well and placed on a rocker with agitation for 24-40 hours at 4° C.

At the end of the incubation, the medium-umbilical cord suspension was centrifuged at 3,000 rpm for 25 minutes. The supernatant was collected. The pellet at the bottom of the tubes was centrifuged at 3,000 rpm for another 25 minutes, and the resulting supernatant was collected.

The supernatants collected from the two centrifugation runs were combined as umbilical cord conditioned medium, which was aliquoted and stored at −80° C., or lyophilized and stored at ambient temperature. The umbilical cord conditioned medium may be used as injectable formula, alone or in combination with other tissues. The umbilical cord conditioned medium is also called micronized umbilical cord conditioned medium or micronized umbilical cord elution.

The umbilical cord particulates in the pellets at the bottom of the tubes following centrifugation were also collected, aliquoted and stored at −80° C. or lyophilized.

### Example 6. Shear Viscosity Measurement of Umbilical Cord Conditioned Medium

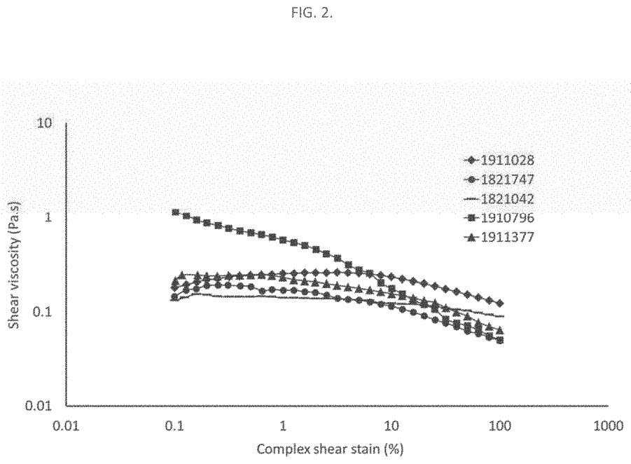

The viscosity of the umbilical cord conditioned medium prepared as in the example 5 was measured by Kinexus lab+ rheometer. The umbilical cord conditioned media (150 ul) was added to the center of a 40 mm roughened plate. Another 40 mm roughened plate was descended to a position where a 0.12 mm gap was maintained between the two plates. Visual confirmation was taken to make sure the gap was completely filled with the umbilical cord conditioned medium. A series of increasing torques was applied to the umbilical cord conditioned media. The shear viscosity of the umbilical cord conditioned medium was then plotted against the strain (FIG. 2).

### Example 7. The Effect of Umbilical Cord Conditioned Media on Cellular Metabolic Activity

The effects of an umbilical cord conditioned medium prepared as in the example 5 on cellular metabolic activities of different cell types, RAW 264.7, human dermal fibroblast, human synoviocyte, were evaluated using Alamar blue (Biorad, BUF012B) dye. Different cell densities and the umbilical cord conditioned medium diluted at different ratios were investigated.

The cells in appropriate cell culture media were seeded at different densities (6250, 12500, 18750, or 25000 cells/cm²) in culture plates, and incubated for a day. On the next day, aliquots of the umbilical cord conditioned media were thawed and further centrifuged at 10,000 rpm for 10 mins to remove debris before being diluted at different ratios (1:5, 1:10, 1:20, 1:30, 1:50 v/v) with cell culture media appropriate for the cells and used to replace the culture media for the cells in the culture plates. The cells were incubated with the umbilical cord conditioned media for 24 hours.

At the end of the treatment, the Alamar blue dye was diluted in culture media appropriate to the cells to a final concentration of 10% (v/v). The Alamar blue containing media were used to replace the umbilical cord conditioned media in the culture plates. The treated cells were then incubated in the Alamar blue containing media for 4-4.5 hours.

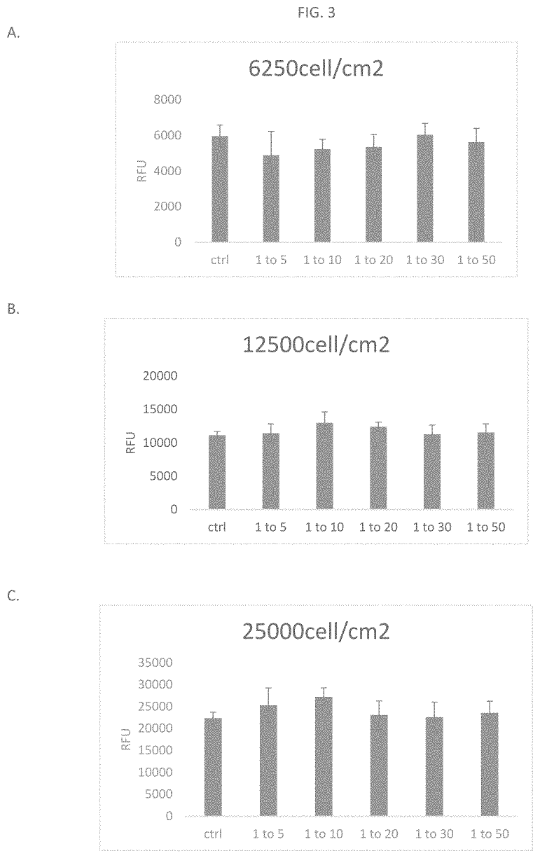

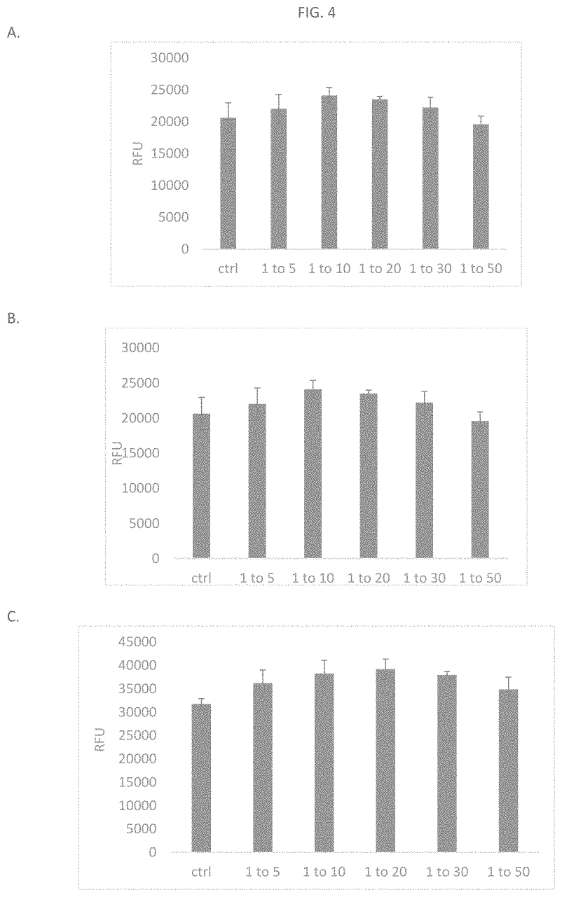

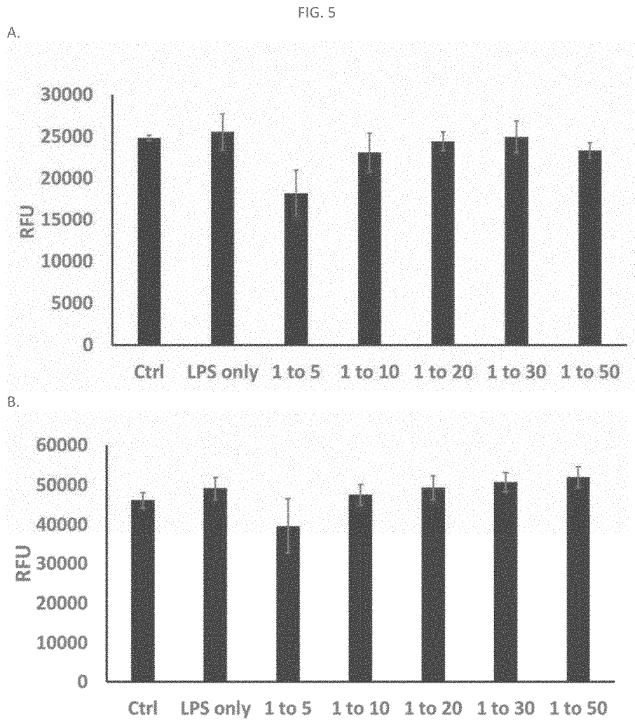

Then, the Alamar blue containing media were collected and transferred to a black, clear bottom 96 well plate to be read by a fluorescence microplate reader to determine the cellular metabolic activities as normalized by a blank Alamar blue reagent for human synoviocyte (FIG. 3), human dermal fibroblast (FIG. 4) and RAW 264.7 (FIG. 5). The results showed that the umbilical cord conditioned medium diluted at 1:10 induced a metabolic increase of 16% and 21% in synoviocytes at 12500 and 25000 cells/cm², respectively. The umbilical cord conditioned media diluted at 1:10 also induced a metabolic increase of 16%, 24%, and 20% in dermal fibroblasts at 12500, 18750, and 25000 cells/cm², respectively. The umbilical cord conditioned media diluted at 1:5 induced a 15% metabolic decrease in RAW cells at 25000 cells/cm².

### Example 8. Anti-Inflammatory Effects of Umbilical Cord Conditioned Medium

The anti-inflammatory effects of the umbilical cord conditioned medium prepared as in the example 5 on inhibition of TNF-alpha secretion by RAW 264.7 cells, a murine macrophage cell line, were studied. TNF-alpha elisa kits (ThermoFisher, BMS607-2INST) were used to determine the TNF-alpha secretion levels from the RAW 264.7 cells after being treated with an umbilical cord conditioned medium diluted at different ratios.

Method 1: Cells were seeded in an appropriate culture medium in culture plates and incubated for a day prior to the treatment with the umbilical cord conditioned medium. On the next day, aliquots of the umbilical cord conditioned medium were thawed and centrifuged at 10,000 rpm for 10 mins to remove debris before being diluted at different ratios (1:5, 1:10, 1:20, 1:30, and 1:50) in a culture medium appropriate for the cells and used to replace the culture medium in the culture plate. Cells were incubated in the umbilical cord conditioned medium diluted at different ratios for 24 hours.

Then, lipopolysaccharide (LPS, Sigma, 5293-2 mL) was added to the culture plates at a final concentration of 1 μg/mL to stimulate TNF-alpha secretion by the cells.

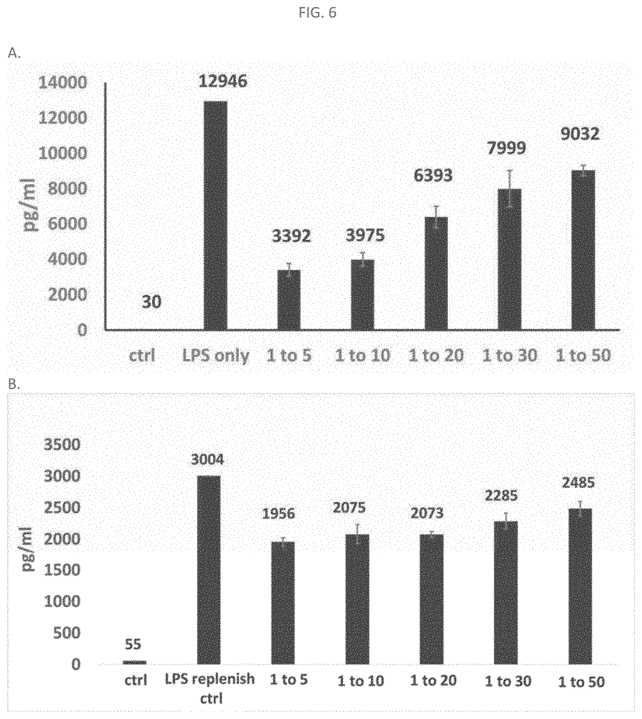

Twenty-four hours following the LPS stimulation, a supernatant from the culture plates was collected and frozen in −80° C. for storage until the TNF-alpha ELISA assay. The measurement of TNF-alpha secretion was done following instructions of the TNF-alpha ELISA kit. As shown in FIG. 6A, the umbilical cord conditioned media effectively inhibited TNF-alpha secretion by the RAW cells in a dose-dependent manner.

Method 2: To mimic the inflammatory responses in vivo, RAW 264.7 cells were seeded in an appropriate culture medium in culture plates and incubated for a day, then stimulated with LPS at a final concentration of 1 ug/ml the next day. Twenty-four hours after LPS stimulation, LPS-containing media in the culture plate was replaced with the umbilical cord conditioned medium diluted at different ratios combined with LPS solution at 1 ug/ml final concentration. Cells were cultured for another 24 hours and the cell culture supernatant from the culture plates was collected and frozen in −80° C. for storage until the TNF-alpha ELISA assay. The measurement of TNF-alpha secretion was done following instructions of the TNF-alpha ELISA kit. As shown in FIG. 6B, the umbilical cord conditioned media effectively inhibited RAW cell TNF-alpha secretion by 18%-35% in a dose-dependent manner.

### Example 9. Revitalization of Cryopreserved Umbilical Cord and Viability Assessment

Cryopreserved umbilical cords as prepared in Example 2 were retrieved from the −80° C. freezer and thawed in a 37° C. water bath with agitation. Thawed umbilical cords were first transferred to a 175 mL falcon tube with 100 mL of the DMEM medium and centrifuged at 1,000 rpm for 5 mins at room temperature. The resulting supernatant was then decanted and another 100 mL of the fresh DMEM medium was added to the 175 mL falcon tube. The umbilical cords were then centrifuged at 1,000 rpm for another 5 mins at room temperature.

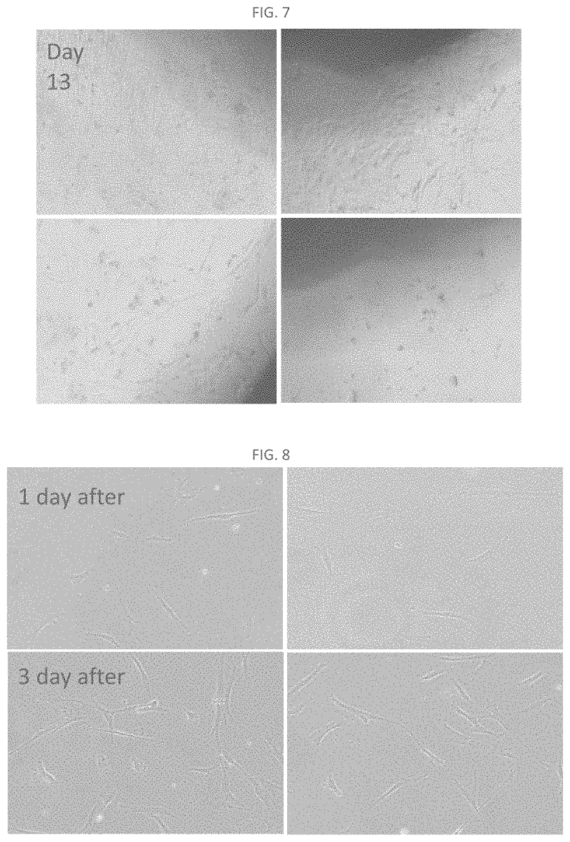

Umbilical cord cell outgrowth: At the end of the second centrifugation, the umbilical cords were placed in a culture petri dish with Wharton's jelly side facing down in an incubator at 37° C. for 1 hour for attachment. An umbilical cord cell culture medium (DMEM containing 1% penicillin/streptomycin, 15% fetal bovine serum, 1% GlutaMAX™) was then added to the petri dish. The umbilical cord culture medium was changed every 2-3 days to revitalize the cryopreserved umbilical cord. Abundant umbilical cord cell in vitro outgrowth from the attached umbilical cord tissue was observed between Day 10 and Day14. The umbilical cord cell outgrowth from the cryopreserved umbilical cords is shown in FIG. 7. The outgrowth cells were detached from the petri dish by 0.05% Trypsin/EDTA (GIBCO, 25300-062) and re-plated into new culture flasks for further expansion (FIG. 8). The outgrowth cells from both fresh umbilical cord and revitalized cryopreserved umbilical cords were expanded for cryopreservation and flow cytometry analysis.

Enzymatic digestion and cell isolation: At the end of the second centrifugation, the umbilical cords were minced into fine pieces and incubated with 2 mg/mL collagenase (GIBCO, 17-100-017) and 1 mg/mL hyaluronidase (Sigma, H3506) dissolved in Hank's balanced salt buffer (GIBCO, 14025-076) at 37° C. on a rocker with gentle agitation (75-85 rpm) for 4-5 hours. At the end of tissue digestion, the tissue/cell suspensions were filtered through a 40 μm cell strainer (Falcon, 352340). An equal amount of a 10% fetal bovine serum (FBS) containing culture medium was added to each filtered tissue/cell suspension and then centrifuged at 1,000 rpm for 5 mins. The supernatant was decanted and the cell pellet was re-suspended in a 10% FBS containing medium and centrifuged for another 5 mins at 1,000 rpm. The supernatant was decanted and the cell pellet was re-suspended in the umbilical cord cell culture medium and plated in tissue culture plates for cell expansion.

### Example 10. Flow Cytometry of Cells Expended from Fresh Umbilical Cord and Cryopreserved Umbilical Cord

Expanded cells prepared from example 9 were used for flow cytometry to characterize the surface marker (CD29, CD44, CD73, CD105, CD166, CD14, CD31, CD34, CD45, and CD19) expressions of the cell populations.

Cells were detached from the culture flasks with 0.05% trypsin/EDTA and neutralized with 10% FBS containing media. The resulting cell suspension was centrifuged at 1,000 rpm for 5 minutes. The supernatant was decanted and cell pellet was re-suspended in a fresh DMEM. A small aliquot of the cell suspension was taken for cell count and the rest of the cell suspension was centrifuged for another 5 minutes at 1,000 rpm. Next, cell pellet was re-suspended in flow cytometry buffer (Invitrogen, 04-4222-57) and centrifuged at 1,000 rpm for 5 mins. The cell pellet was re-suspended in flow cytometry buffer at a density not less than 250,000 cells/mL of buffer.

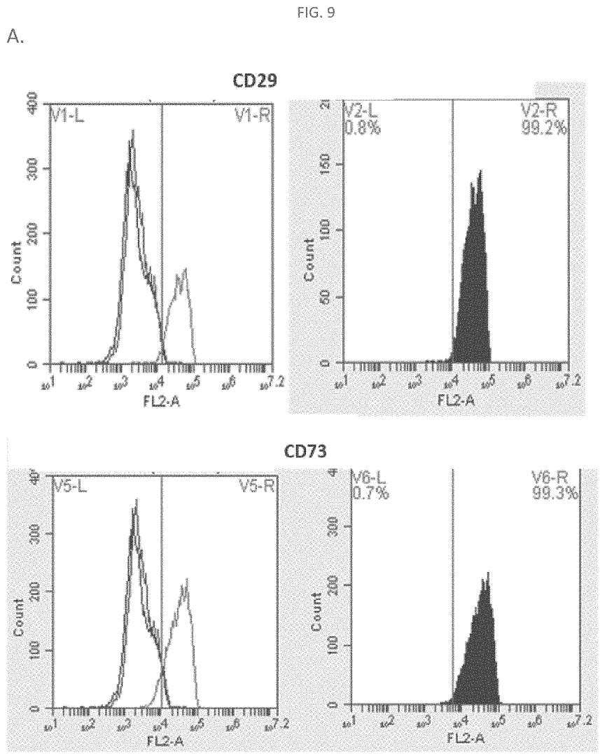

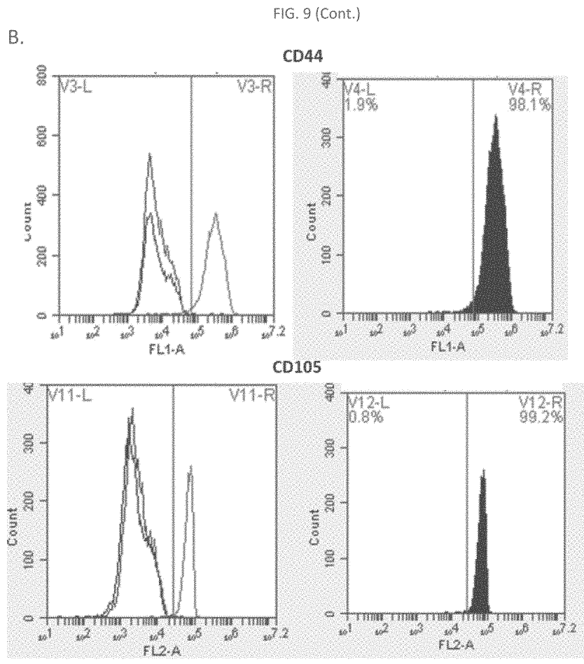

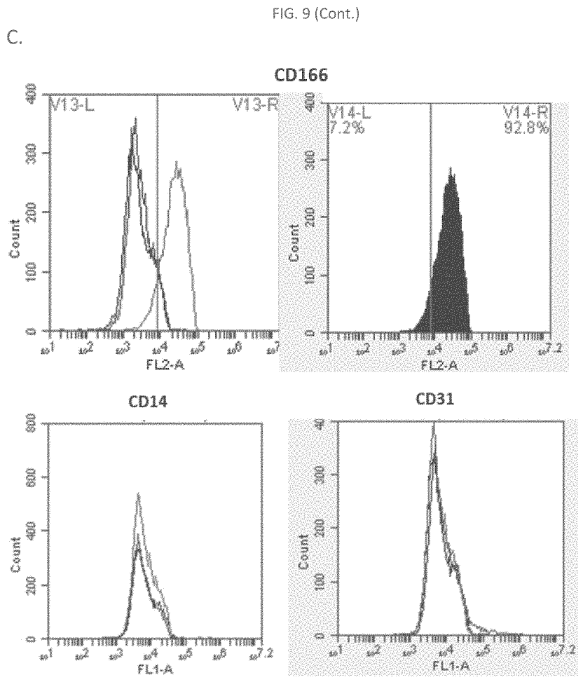

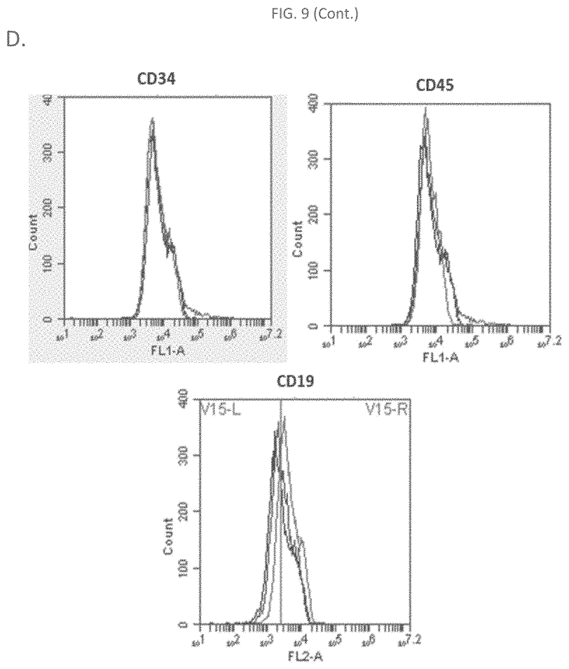

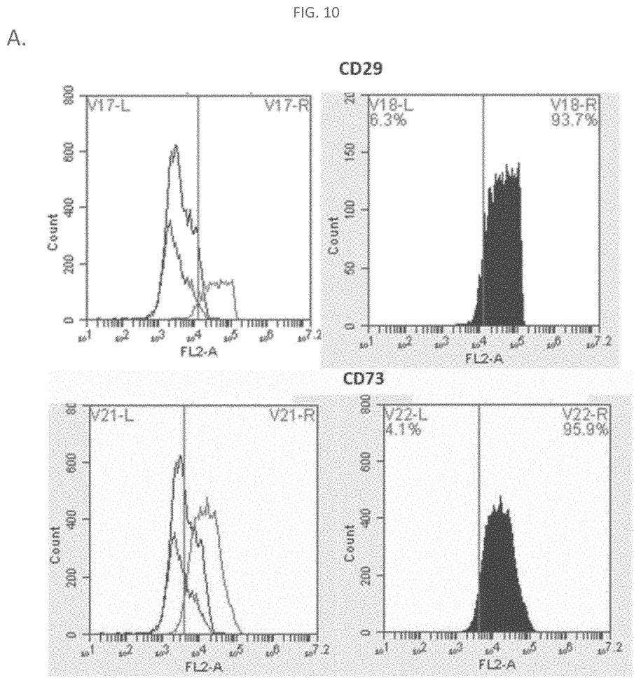

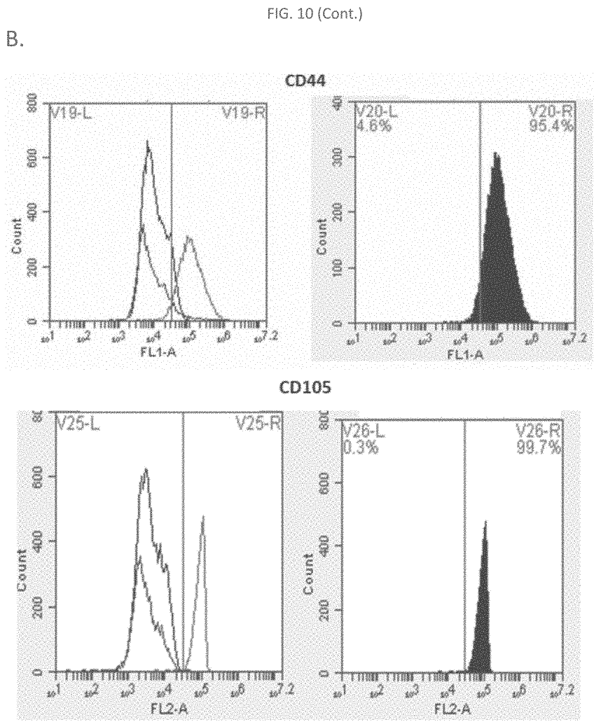

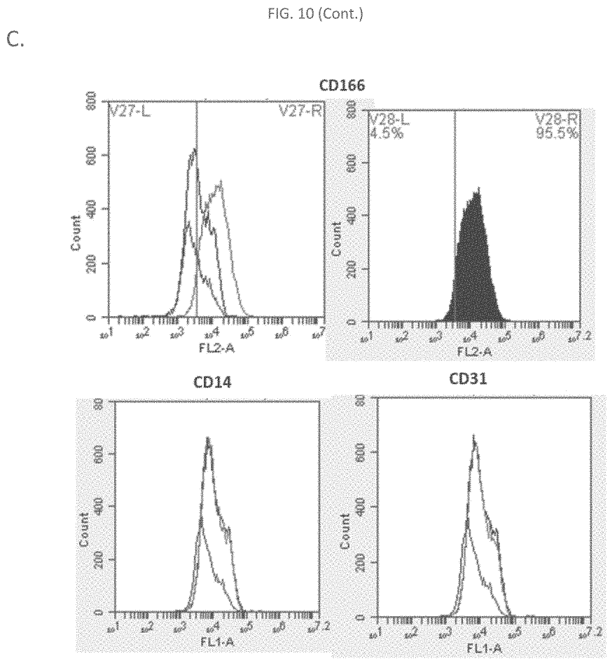

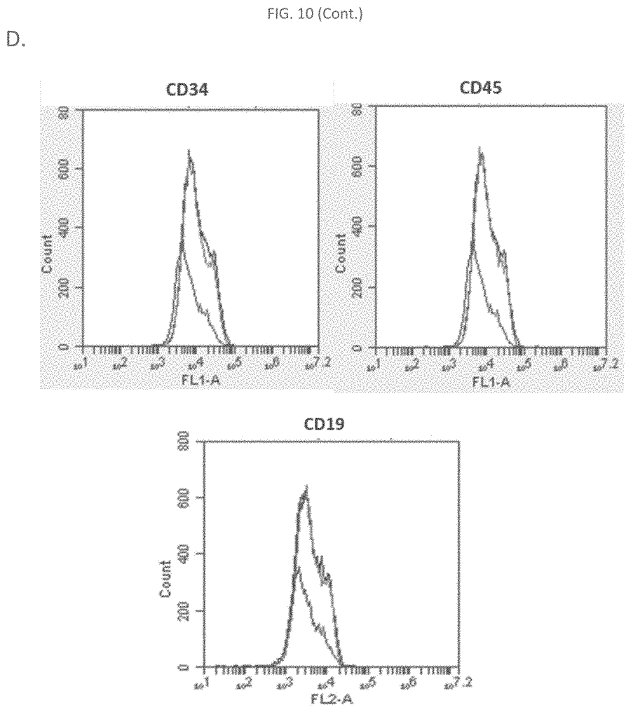

Aliquots of 200 μL cell suspension were pipetted to corresponding wells in a 96 well plate and flow cytometry antibodies were added at 1:200 dilution to each well. The cells were incubated with the antibodies for 1 hour at 4° C. and protected from light. At the end of the antibody incubation, an additional 100 μL flow cytometry buffer was added to each well and the 96 well plate was centrifuged at 1,500 rpm for 3 mins. The supernatant was decanted from each well and another 300 μL of flow cytometry buffer was added to each well to rinse the cells. The plate was centrifuged at 1,500 rpm for another 3 mins. The supernatant was decanted and the cells from each well were re-suspended in 200 μL flow cytometry buffer with the addition of a cell viability dye and then transferred to a 1.5 mL Eppendorf tube for flow cytometry analysis. Appropriate isotype control for each antibody was also performed. At least 10,000 events were collected for each analysis. The marker expression of the outgrowth cells from fresh umbilical cord were shown in FIG. 9. The results of the revitalized cells outgrown from the cryopreserved umbilical cords were shown in FIG. 10. The data showed that both cell populations have similar surface marker expression profiles.

### Example 11. Preparation of Placental Membrane Particulates

Human term placenta and amniotic sac with research consent was obtained after caesarean section and transferred to the processing facility under sterile condition.

The amniotic sac comprising placental membrane with both amniotic and chorionic membrane layers were cut around the placenta skirt and rinsed at least three times with isotonic solution such as saline, or Lactated Ringer to remove loosely bond blood. The rinsed placental membrane with both amnion and chorion layers in contact was laid on a sterile board with amniotic membrane epithelia layer facing up. Different sizes and shapes of mesh frames were laid on top of the placental membrane. The placental membrane was cut to different sizes and shapes aligned with the sizes and shapes of the matching frames. The placental membrane pieces were rinsed with isotonic saline for three times with agitation, five minutes each time, followed by decellularization for 2-5 hours with agitation at ambient temperature. The decellularized placental membrane was rinsed with isotonic solution or water followed by or lyophilization or storage at −80° C.

Lyophilized decellularized placental membrane: The lyophilized placental membrane was micronized in Retch mill (ZM200) to generate micronized placental membrane, also called placental membrane particulates. The placental membrane particulates were collected, sieved to different size range with a tap sifter, and weighed. The placental membrane particulates were aliquoted and stored at ambient temperature for further preparation and different characterization assessment.

Frozen decellularized placental membrane: The frozen decellularized placental membrane was cut into small pieces (˜0.5 cm). A cryomill (Retsch) or a grinder was used to micronize the small frozen decellularized placental membrane pieces. The frozen decellularized placental membrane pieces were transferred to a grinding jar with a grinding ball, and then the jar was sealed. The grinding jar was pre-cooled by liquid nitrogen prior to the grinding process. The frozen decellularized placental membrane pieces were pulverized for 15 mins at 30 Hz with the grinding jar being continually cooled with liquid nitrogen. The resulted frozen particulates were transferred to sterile 50 mL conical tubes from the grinding jar, thawed and centrifuged at 3,000 rpm for 5 mins to collect the placental membrane particulates at the bottom of the tubes. The placental membrane particulates were then aliquoted and stored at −80° C. or lyophilized.

### Example 12. Umbilical Cord Conditioned Medium Hydrogel

The lyophilized umbilical cord particulates at 10 mg dry weight from example 5 was treated with 1 mg pepsin in 0.01N HCl (1 mL) with agitation at room temperature for 48 hrs. Following pepsin treatment, the umbilical cord particulate digest was either aliquoted for long term storage at −80° C. freezer or utilized to prepare umbilical cord conditioned medium hydrogel.

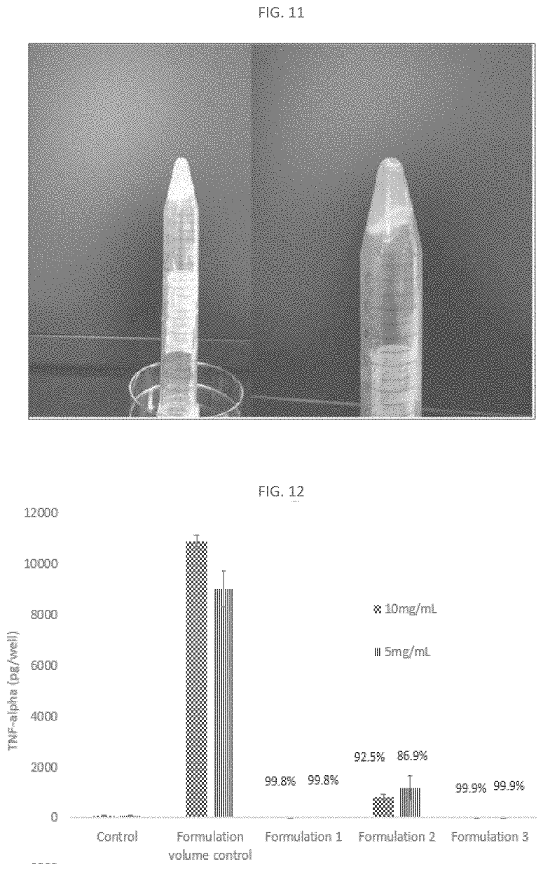

The umbilical cord particulate digest (10 mg/ml) was first neutralized with one-tenth the digest volume of 0.1 N NaOH and one-ninth the digest volume of 10×PBS. Neutralized pre-gel solution was then diluted to the desired final concentration with umbilical cord conditioned medium and placed at 37° C. for 30-45 mins for hydrogel formation. FIG. 11 shows a 6 mg/ml umbilical cord conditioned medium hydrogel.

### Example 13. Preparation of Birth Tissue Injectable Compositions

Various birth tissue injectable mixtures were prepared with the umbilical cord conditioned medium of Example 5 and/or Example 12 with (1) the placental membrane particulates of Example 11, (2) the umbilical cord particulates of Example 3, (3) the cryopreserved umbilical cord particulates of Example 4, or (4) the umbilical cord pellets of Example 5, in combination with the umbilical cord particulates of Example 3 or the cryopreserved umbilical cord particulates of Example 4.

Method 1: The umbilical cord conditioned medium prepared in Example 5 was used as it is or diluted at different ratios (1:2, 1:5, 1:10, 1:20, 1:30, 1:40, or 1:50, v/v) in an appropriate solution such as water, saline, DMEM, or DPBS. Different volumes of the undiluted or diluted umbilical cord conditioned media was transferred into the placental membrane particulates as prepared in Example 11 to generate different volume to dry weight ratios. The placental membrane particulates were mixed with the solution completely by pipetting, vortexing and inversion. The resulting birth tissue mixture may be aliquoted again, centrifuged to package and lyophilized.

Method 2: The umbilical cord conditioned medium prepared in Example 5 was used as it is or diluted at different ratios (1:2, 1:5, 1:10, 1:20, 1:30, 1:40, or 1:50, v/v) in an appropriate solution such as water, saline, DMEM, or DPBS. Different volumes of the undiluted or diluted umbilical cord conditioned media was transferred into the umbilical cord particulates prepared in Example 3. The particulates were mixed with solution completely by pipetting, vortexing and inversion. The resulting birth tissue mixture may be aliquoted again, centrifuged to package and lyophilized.

Method 3: The umbilical cord conditioned medium prepared in Example 5 was used as it is or diluted at different ratios (1:2, 1:5, 1:10, 1:20, 1:30, 1:40, or 1:50, v/v) in an appropriate solution such as water, saline, DMEM, or DPBS. Different volumes of the undiluted or diluted umbilical cord conditioned media was transferred into the umbilical cord particulates prepared in Example 5. The particulates were mixed with the solution completely by pipetting, vortexing and inversion. The resulting birth tissue mixture may be aliquoted again, centrifuged to package and lyophilized.

Method 4: The cryopreserved particulates from example 4 are thawed at 37° C., mixed with an appropriate solution such as water, saline, DMEM, or DPBS and centrifuged to rinse off the cryopreservation media. Then the particulates are mixed with the umbilical cord conditioned medium prepared in Example 5 as it is or diluted at different ratios (1:2, 1:5, 1:10, 1:20, 1:30, 1:40, or 1:50, v/v) in an appropriate solution such as water, saline, DMEM, or DPBS. The mixture may be injected to the injured body parts.

Method 5: The umbilical cord conditioned medium prepared in Example 5 was used as it is or diluted at different ratios (1:2, 1:5, 1:10, 1:20, 1:30, 1:40, or 1:50, v/v) in an appropriate solution such as water, saline, DMEM, or DPBS. Different volumes of the undiluted or diluted umbilical cord conditioned media was transferred into the placental membrane particulates aliquots prepared in Example 11 and the umbilical cord particulates prepared in Example 3. The particulates were mixed with solution completely by pipetting, vortexing and inversion. The resulting birth tissue mixture may be aliquoted again, centrifuged to package and lyophilized.

Method 6: The cryopreserved particulates from example 4 will be thawed at 37° C., then mixed with an appropriate solution such as water, saline, DMEM, or DPBS and centrifuged to rinse off the cryopreservation media. Then the particulates are mixed with the umbilical cord conditioned medium prepared in Example 5 that are either undiluted or diluted at different ratios (1:2, 1:5, 1:10, 1:20, 1:30, 1:40, or 1:50, v/v) in an appropriate solution such as water, saline, DMEM, or DPBS and the placental membrane particulates aliquots prepared in Example 11. The mixture may be injected to the injured body parts.

### Example 14. Characterization of Birth Tissue Injectable Compositions

The birth tissue injectable compositions prepared in Example 13 will be characterized.

Injectability assessment: Injectable formulas prepared with the umbilical cord particulates of Example 3, the cryopreserved umbilical cord particulates of Example 4, the umbilical cord conditioned medium of Example 5, the umbilical cord pellets of Example 5, the placental membrane particulates of Example 11, the umbilical cord conditioned media hydrogel of example 12, or the injectable mixtures of Example 13 were tested using syringes with different gauge needles.

Component assessment: The umbilical cord conditioned medium of Example 5 was quantified for its contents of hyaluronic acid and cytokines. The hyaluronic acid in the umbilical cord conditioned medium was quantified by hyaluronic acid ELISA kits (Hyaluronan Quantikine ELISA Kit, R&D systems). The umbilical cord conditioned medium was diluted at a ratio of 1:200,000 in a DMEM with 1% antibiotics or ELISA kit assay diluent for quantification. The hyaluronic acid content was quantified according to the manufacturer's protocol and the average hyaluronic acid quantity from 4 donors was 1.68±0.28 mg/mL elution. The amounts of IL-1RA (Interlukin 1 receptor antagonist) and TIMP1 (Tissue inhibitor of metalloproteinase 1) in the umbilical cord conditioned medium were quantified using IL-1RA Simplex kit (EPX01A-12080-901, Thermofisher Scientific), TIMP1 Simplex kits (EPX01A-12018-901, Thermofisher Scientific) and human basic kits (EPX010-10420-901, Thermofisher Scientific) with Luminex 200 (R&D systems). The umbilical cord conditioned medium was diluted at 1:20 for the IL-1RA quantification and 1:426 for the TIMP1 quantification, respectively, in a DMEM with 1% antibiotics. IL-1RA and TIMP1 were quantified according to the manufacturer's protocols and the average quantity of IL-1RA and TIMP1 from 4 donors were 312±153 ng/mL elution and 4±1.9 μg/mL elution, respectively.

The different injectable formulas with placental membrane particulates, umbilical cord particulates, and umbilical cord conditioned medium were extracted with a protease (collagenase type I) and hyaluronidase. The quantity of different growth factors, cytokines, and other macromolecules were measured using ELISA or multiplex assay for the component assessment. Three different formulations from 3-9 donors, formulation 1 (umbilical cord conditioned medium from example 5+ umbilical cord pellet from example 5+ placental membrane from example 11), formulation 2 (umbilical cord conditioned medium from example 5) and formulation 3 (umbilical cord conditioned medium from example 5+ umbilical cord particulates from example 3+ placental membrane from example 11). The data is summarized in Table 1.

Activity assessment: Different injectable mixtures or formulas were added to various cell culture media. Cell proliferative activity, MMP1 inhibition, and anti-inflammatory activity were tested.

Anti-Inflammatory Effect:

The anti-inflammatory effects of the injectable birth tissue formulations on inhibition of TNF-alpha secretion by RAW 264.7 cells, a murine macrophage cell line, were studied. Three different formulations from 3 donors, formulation 1 (umbilical cord conditioned medium from example 5+ umbilical cord pellet from example 5+ placental membrane from example 11), formulation 2 (umbilical cord conditioned medium from example 5) and formulation 3 (umbilical cord conditioned medium from example 5+ umbilical cord particulates from example 3+ placental membrane from example 11), were prepared.

TNF-alpha elisa kits (ThermoFisher, BMS607-2INST) were used to determine the TNF-alpha secretion levels from the RAW 264.7 cells after being treated with the injectable birth tissue formulations.

Cells were seeded in an appropriate culture medium in culture plates and incubated for a day prior to the treatment with the injectable birth tissue formulations. On the next day, aliquots of the injectable birth tissue formulations were prepared at 2 different concentrations, 10 mg/mL and 5 mg/mL, in a culture medium appropriate for the cells and used to replace the culture medium in the culture plate. Cells were incubated in the presence of the injectable birth tissue formulations for 24 hours. Then, lipopolysaccharide (LPS, Sigma, 5293-2 mL) was added to the culture plates at a final concentration of 1 μg/mL to stimulate TNF-alpha secretion by the cells.

Twenty-four hours following the LPS stimulation, a supernatant from the culture plates was collected and frozen in −80° C. for storage until the TNF-alpha ELISA assay. The measurement of TNF-alpha secretion was done following instructions of the TNF-alpha ELISA kit. As shown in FIG. 12, the injectable birth tissue formulations effectively inhibited TNF-alpha secretion by the RAW cells in a dose-dependent manner. All three injectable birth tissue formulations effectively reduced the TNF-alpha secretion from LPS stimulated RAW cells. More than 99% TNF-alpha reduction was seen in the RAW cells treated by the formulation 1 and formulation 3 at both concentrations tested when compared to the formulation volume control group. Formulation 2 at 10 mg/mL and 5 mg/mL resulted in a dose-dependent RAW cell TNF-alpha reduction of 92.5% and 86.9%, respectively, when compared to the formulation volume control group.

Proliferative Effect:

The proliferative effects of the injectable birth tissue formulations on primary human synoviocyte were studied. Three different formulations from 3 donors, formulation 1 (umbilical cord conditioned medium from example 5+ umbilical cord pellet from example 5+ placental membrane from example 11), formulation 2 (umbilical cord conditioned medium from example 5) and formulation 3 (umbilical cord pellet from example 5), were prepared.

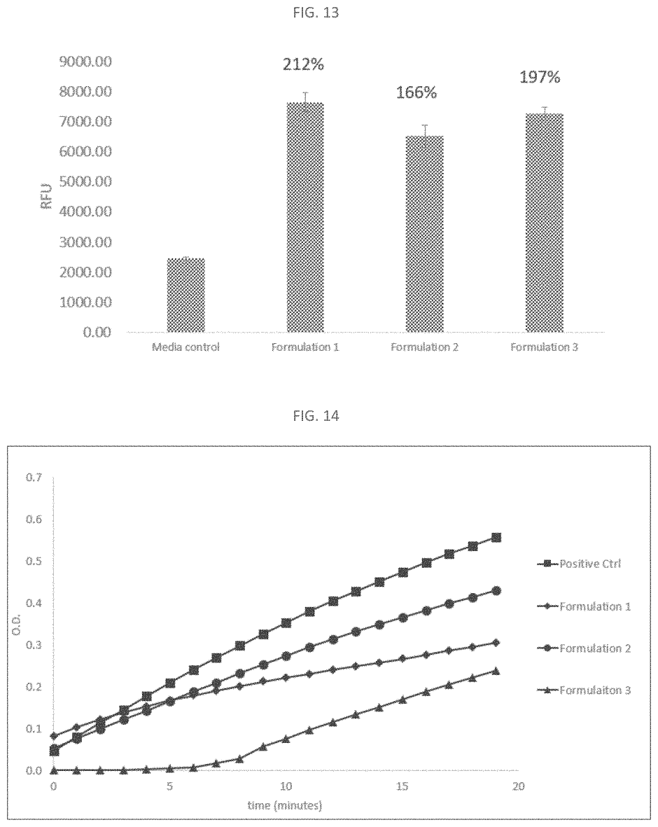

Cells were seeded in an appropriate culture medium in the bottom wells of an insert culture plates and incubated for a day prior to the treatment with the injectable birth tissue formulations. On the next day, aliquots of the injectable birth tissue formulations were prepared in a culture medium appropriate for the cells and added to the inserts to reach a final concentration of ˜2.2 mg/mL. Cells were incubated in the presence of the injectable birth tissue formulations for 4 days. At Day4, 10% alamar blue reagent in appropriate cell culture media was used to measure the metabolic activities of the cells and fluorescent readings were used as a representation of relative cell numbers between groups. The data showed that all 3 formulation groups effectively induced primary human synovicoyte proliferation when compared to the media control group (FIG. 13).

MMP1 Inhibition Effects:

Three injectable birth tissue formulations from 3 donors, formulation 1 (umbilical cord elute from example 5+ umbilical cord pellet from example 5+ placental membrane from example 11), formulation 2 (placental membrane from example 11), formulation 3 (umbilical cord umbilical cord particulates from example 3), were prepared. The formulations were pre-incubated with MMP1 enzymes (50 ng/mL) for 2 hrs mins at 37° C. Then the injectable birth tissue formulation-MMP1 enzyme suspensions were centrifuged at 10,000 RPM for 1 mins. The supernatant was collected, added to a 96 well plate and mixed with the MMP1 enzyme substrate. Dynamic absorbance readings were performed and a higher O.D. value corresponds to a higher MMP1 enzyme activity. The results showed that all three injectable birth tissue formulations tested inhibited MMP1 enzyme activity (FIG. 14).

### Example 15. Shear Viscosity Measurement of Injectable Birth Tissue Formulations with or without Umbilical Cord Conditioned Media

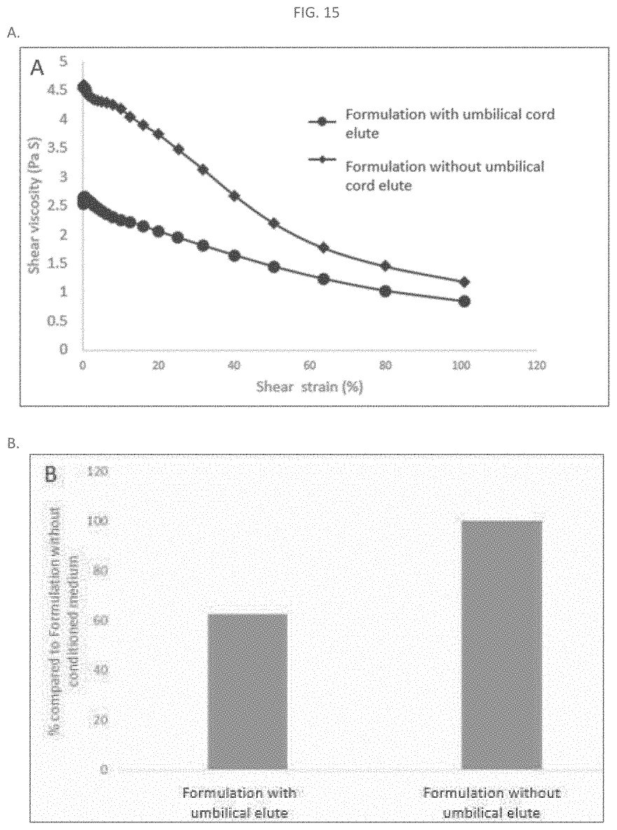

Two injectable birth tissue formulations were prepared by mixing the umbilical cord particulate pellets prepared as in the Example 5 and the decellularized placenta membrane particulate prepared as in the Example 11 with or without the addition of umbilical cord conditioned medium prepared as in the Example 5. The shear viscosity of the injectable birth tissue formulations was measured by Kinexus lab+ rheometer. The injectable birth tissue formulations (150 ul) was added to the center of a 40 mm roughened plate. Another 40 mm roughened plate was descended to a position where a 0.12 mm gap was maintained between the two plates. Visual confirmation was taken to make sure the gap was completely filled with the injectable birth tissue formulations. A series of increasing torques was applied. The shear viscosity of the injectable birth tissue formulation was then plotted against the shear strain (FIG. 15A). Formulation with umbilical cord conditioned medium consistently showed lower shear viscosity when compared to the formulation without umbilical cord conditioned medium. An average of 38% reduction, from 3 donors, in shear viscosity from the formulation with umbilical cord conditioned medium was observed when compared to the formulation without umbilical cord conditioned medium at 50% shear strain (FIG. 15B). The results showed that the umbilical cord conditioned medium was able to reduce the shear viscosity of injectable birth tissue formulations.

### Example 16. Cohesiveness Test of Injectable Birth Tissue Formulations with or without Umbilical Cord Conditioned Media

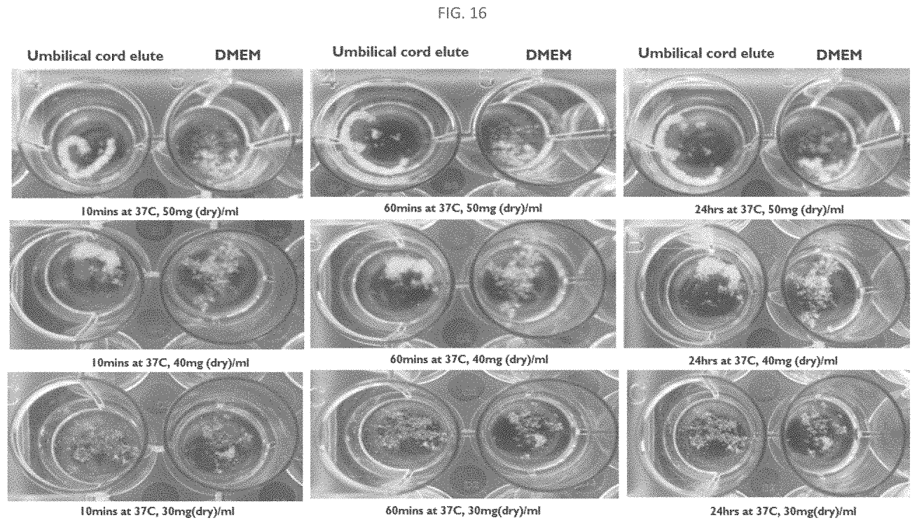

Lyophilized placental membrane (PM) particulates prepared as in the Example 11 were resuspended in either umbilical cord conditioned medium prepared from Example 5 from 1 donor or Dulbecco's Modified Eagle's Medium (DMEM) with 1% antibiotics at 3 different concentrations, 50 mg (dry) particulate/mL, 40 mg (dry) particulate/mL and 30 mg (dry) particulate/mL. Then 50 ul of the resuspended injectable PM particulates were added to 500 ul sterile saline in a 24 well plate using a p-200 ul pipette. The plate was incubated at 37° C. and pictures of injectable PM formulations were taken at, 10 mins, 60 mins and 24 hours post-incubation. The results showed that, at a concentration of 40 mg (dry) particulate/mL and above, the umbilical cord conditioned medium was able to enhance the cohesiveness of the injectable PM particulates (FIG. 16).

### Example 17. MMP1 Enzyme Activity Inhibition by Injectable Birth Tissue Formulations with or without Umbilical Cord Conditioned Media

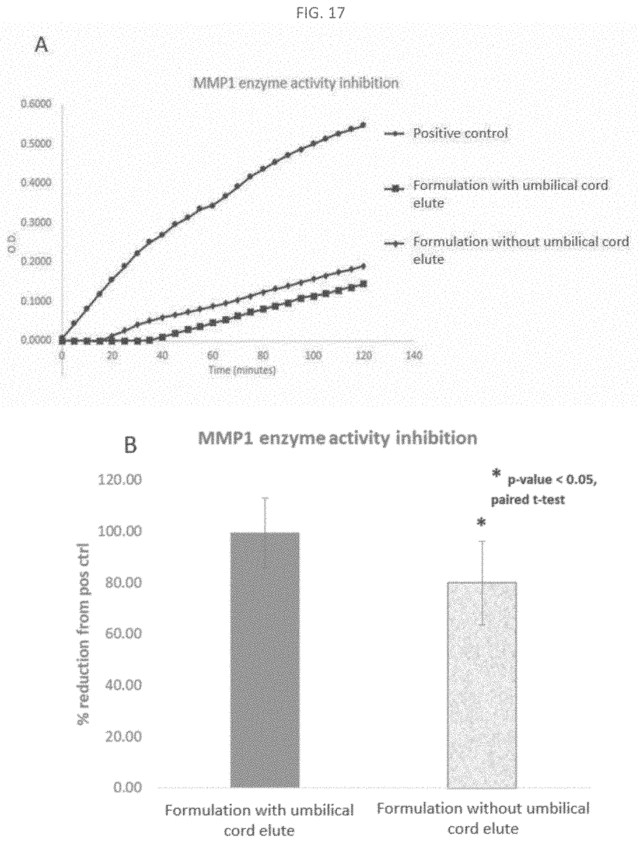

Two injectable birth tissue formulations, from 4 donors, were prepared by mixing the umbilical cord particulates prepared as in the Example 3 and the decellularized placenta membrane particulates prepared as in the Example 11 with or without the addition of umbilical cord conditioned medium prepared as in the Example 5. The formulations were pre-incubated with MMP1 enzymes (50 ng/mL) for 5 mins at 37° C. Then the injectable birth tissue formulation-MMP1 enzyme suspensions were centrifuged at 10,000 RPM for 5 mins. The supernatant was collected, added to a 96 well plate and mixed with the MMP1 enzyme substrate. Dynamic absorbance readings were performed and a higher O.D. value corresponds to a higher MMP1 enzyme activity. The results showed that the injectable birth tissue formulations inhibited MMP1 enzyme activity, while the formulation with umbilical cord conditioned media inhibited MMP1 activity more than formulation without conditioned media from 25 minutes to 120 minutes (FIG. 17A). Moreover, at minute 35, the injectable birth tissue formulation with umbilical cord conditioned medium demonstrated statistically significantly better inhibitory effects than the injectable birth tissue formulation without umbilical cord conditioned medium (FIG. 17B).

### Example 18. Biochemical Factor Time Course Release Assay of Injectable Birth Tissue Formulations with or without Umbilical Cord Conditioned Media

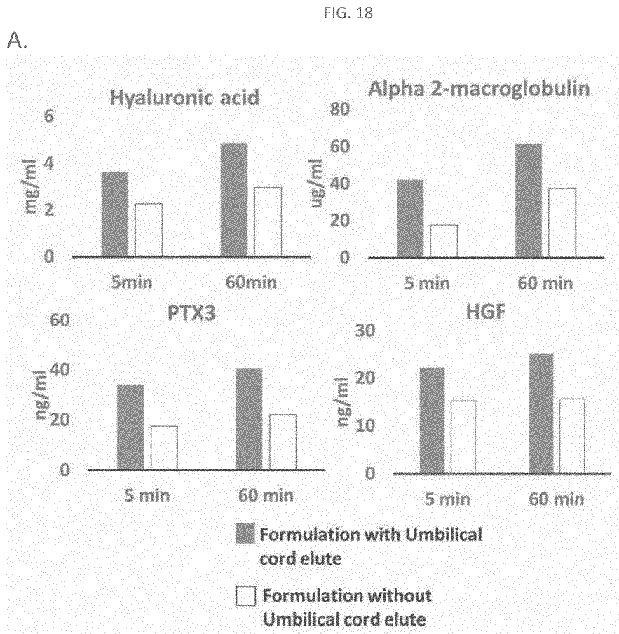

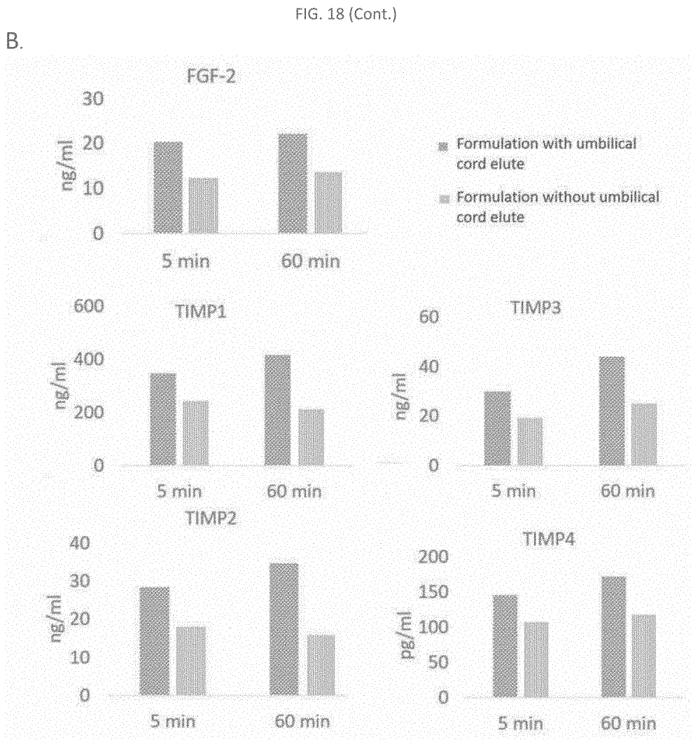

Two injectable birth tissue formulations, from 2-3 donors, were prepared by mixing the umbilical cord particulates prepared as in the Example 3 and the decellularized placenta membrane particulate prepared as in the Example 11 with or without the addition of umbilical cord conditioned medium prepared as in the Example 5. The formulations were rehydrated at a concentration of 50 mg (dry) particulate/mL with sterile saline and mixed well at ambient temperature. After 5 minutes or 60 minutes at ambient temperature, aliquots were taken from different hydrated formulations. Aliquots were then centrifuged at 12,000 RPM for 2 mins. Supernatants were collected and stored at −80° C. until further analyte concentration quantifications. The results showed that the injectable birth tissue formulation with umbilical cord conditioned medium contained more readily available soluble anti-inflammatory factors and proteases inhibitors at both 5 mins and 60 mins following rehydration when compared to the formulation without umbilical cord conditioned medium (FIG. 18). The percentage increase of each biochemical factor in the formulation with umbilical cord conditioned medium when compared to the formulation without umbilical cord conditioned medium is reported as a range of percentage increase between multiple donors and summarized in Table 2.

### Example 19. Recombinant Fibroblast Growth Factor 2 (FGF-2) Protection Over Heat Degradation by Umbilical Cord Conditioned Medium

### (1) Lyophilized Umbilical Cord Conditioned Medium

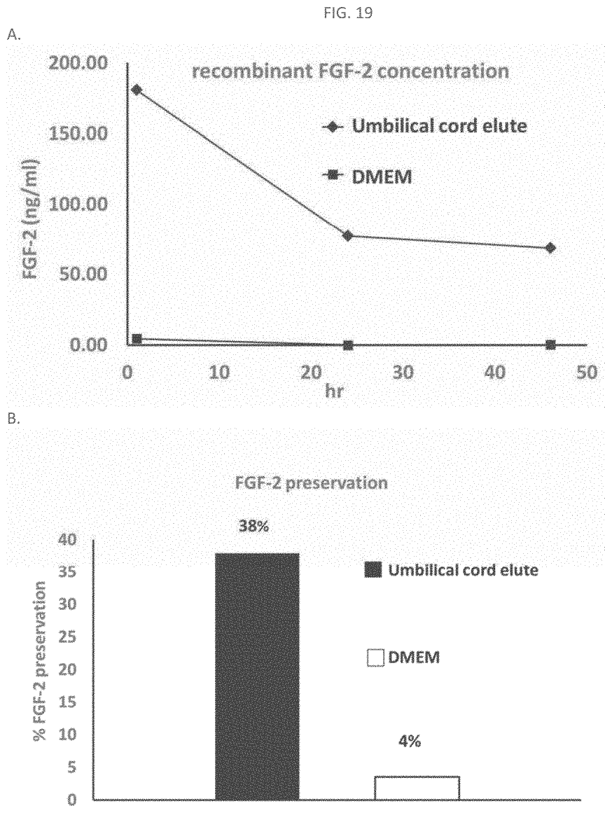

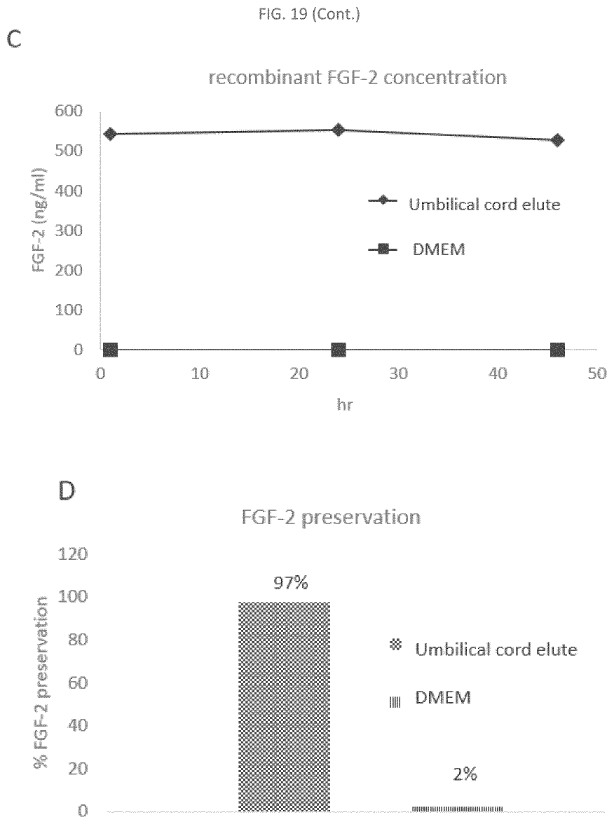

Lyophilized umbilical cord conditioned medium prepared as in the Example 5 from 1 donor was reconstituted with DMEM with 1% antibiotics. Commercially available recombinant FGF-2 was added to the reconstituted umbilical cord conditioned medium (conditioned medium+FGF-2) or DMEM with 1% antibiotics (DMEM+FGF-2) at a final concentration of 400 ng/mL recombinant FGF-2. Umbilical cord conditioned medium (conditioned medium) from the same donor and DMEM with 1% antibiotics (DMEM) were used as the baseline controls, respectively. Four groups were mixed well and incubated at 37° C. Fresh samples (without being frozen) were taken at 1 hr, 24 hrs and 46 hrs following incubation. Commercially available FGF-2 ELISA kits were used to measure the FGF-2 concentrations from each group at each time point. The concentrations of the preserved recombinant FGF-2 in the conditioned medium+FGF-2 and DMEM+FGF-2 groups were obtained by deducting the FGF-2 concentration of each group from the FGF-2 concentration of the corresponding baseline control group at each time point. The data was presented as the recombinant FGF-2 concentration at each time point (FIG. 19A) and the percentage of remaining recombinant FGF-2 concentration at 46 hrs when compared to the recombinant FGF-2 concentration at 1 hr (FIG. 19B). The result showed that reconstituted umbilical cord conditioned medium exhibited a protective effect to the recombinant FGF-2 over heat degradation.

### (2) Frozen Umbilical Cord Conditioned Medium

Frozen umbilical cord conditioned medium prepared as in the Example 5 from 1 donor was thawed and used in the experiment. Commercially available recombinant FGF-2 was added to the umbilical cord conditioned medium (conditioned medium+FGF-2) or DMEM with 1% antibiotics (DMEM+FGF-2) at a final concentration of 400 ng/mL recombinant FGF-2. Umbilical cord conditioned medium (conditioned medium) from the same donor and DMEM with 1% antibiotics (DMEM) were used as the baseline controls, respectively. Four groups were mixed well and incubated at 37° C. Samples were taken at 3 hrs, 24 hrs and 46 hrs following incubation. Commercially available FGF-2 ELISA kits were used to measure the FGF-2 concentrations from each group at each time point. The concentrations of the preserved recombinant FGF-2 in the conditioned medium+FGF-2 and DMEM+FGF-2 groups were obtained by deducting the FGF-2 concentration of each group from the FGF-2 concentration of the corresponding baseline control group at each time point. The data was presented as the recombinant FGF-2 concentration at each time point (FIG. 19C) and the percentage of remaining recombinant FGF-2 concentration at 46 hrs when compared to the recombinant FGF-2 concentration at 3 hr (FIG. 19D). The result showed that frozen umbilical cord conditioned medium exhibited protective effects to the recombinant FGF-2 over heat degradation.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

TABLE 1
Injectable birth tissue bioactive components assessment

| | Formulation 1 | | Formulation 2 | | Formulation 3 | |
|---|---|---|---|---|---|---|
| | Average | Concen- | Average | Concen- | Average | Concen- |
| Analayte | concen- | tration | concen- | tration | concen- | tration |
| (dry weight) | tration | range | tration | range | tration | range |
| HA (ug/mg) | 69.4 | 23-133.6 | 69.4 | 36-104.8 | 116.2 | 62.3-167.3 |
| Alpha2- | 271.5 | 133.4-371.4 | 422.3 | 89-1224.9 | 418.4 | 191.1-739.5 |
| macroglobulin | | | | | | |
| (ng/mg) | | | | | | |
| TIMP1 (pg/mg) | 726.7 | 312-1472 | 5252.2 | 1182-10268 | 606.4 | 445-774 |
| TIMP2 (pg/mg) | 4239.7 | 1448-9635 | 1561.8 | 248-3090 | 2401.7 | 1745-2896 |
| TIMP3 (pg/mg) | 80.5 | 8-182 | 122.4 | 13-289 | 116.0 | 83-179 |
| TIMP4 (pg/mg) | 8.4 | 3.8-16.3 | 3.6 | 0-7.33 | 6.2 | 4.8-8.9 |
| HGF (pg/mg) | 2447.0 | 155-4719 | 131.0 | 49-266 | 4801.1 | 3667-7021 |
| IL-1RA (pg/mg) | 873.4 | 189-1757 | 1477.0 | 186-2864 | 2487.6 | 2153-3139 |
| PTX-3 (pg/mg) | 3400.6 | 25-11504 | 4846.4 | 3083-6431 | 24.0 | 10.9-34.3 |
| TGF-beta 3 (pg/mg) | 205.5 | 99-264 | 116.5 | 60-170 | 438.9 | 304-545 |
| PRG-4 (pg/mg) | 262.7 | 51-615 | 243.0 | 91-778 | 172.1 | 101-284 |
| TGF-beta (pg/mg) | 375.2 | 199.7-497.5 | 249.0 | 32.2-524.9 | 590.8 | 512.8-658.5 |
| bFGF/FGF-2 (pg/mg) | 692.2 | 58-1455 | 25.2 | 16-24 | 1499.6 | 1151-1728 |
| PIGF (pg/mg) | 15.7 | 5.3-33.5 | 4.9 | 3.2-7.3 | 20.1 | 13.6-26.5 |
| VEGF-A (pg/mg) | 160.4 | 83.5-311 | 5.7 | 1.7-10.8 | 130.1 | 78.6-180.6 |
| G-CSF (pg/mg) | 56.3 | 29.5-74.3 | 33.7 | 0-92 | 128.2 | 90-173 |
| MCP-1 (pg/mg) | 113.8 | 51.1-230.1 | 132.2 | 65.5-288.9 | 185.7 | 158.8-224.9 |
| Angiogenin (pg/mg) | 31.9 | 11.3-61.1 | 21.8 | 6.8-45.4 | 74.4 | 50.2-100.3 |
| IL-8 (pg/mg) | 39.1 | 22-52.6 | 28.8 | 20.8-38.2 | 49.1 | 28.4-82.2 |
| EGF (pg/mg) | 4.4 | 2.5-6.5 | un- | un- | 6.8 | 6.6-6.9 |
| | | | detectable | detectable | | |
| PDGF-BB (pg/mg) | 302.7 | 202.9-397.7 | un- | un- | 449.0 | 427.8-476.1 |
| | | | detectable | detectable | | |

TABLE 2
Percentage increase of readily available soluble biochemical factors in the
formulation with umbilical cord elute when compared to the formulation without umbilical
cord elute

| % | | | | | | | | | |
|---|---|---|---|---|---|---|---|---|---|
| increase vs. | | | | | | | | | |
| formulation | | | | | | | | | |
| without | Hyalu- | Alpha2- | | | | | | | |
| umbilical | ornic | macro- | | | | | | | |
| cord elute | acid | globulin | PTX-3 | HGF | FGF-2 | TIMP1 | TIMP2 | TIMP3 | TIMP4 |
| 5 mins | 44-105% | 55-140% | 45-140% | 15-163% | 11-109% | 42-97% | 36-92% | 29-101% | 36-101% |
| 60 mins | 13-125% | 64-456% | 81-282% | 62-145% | 61-64% | 96-151% | 57-118% | 36-75% | 26-47% |

1-202. (canceled)

203. A composition for treating a pathological condition in a body part of a patient in need thereof, comprising an effective amount of an elute of a first birth tissue and a pharmaceutically acceptable carrier.

204. The composition of claim 203, further comprising particulates of a second birth tissue.

205. The composition of claim 204, further comprising particulates of the first birth tissue.

206. The composition of claim 203, wherein the first birth tissue is selected from the group consisting of an umbilical cord, an amniotic sac, a placental plate, a placental membrane and a combination thereof, wherein the placental membrane is from the amniotic sac and comprises amniotic membrane, chorionic membrane and trophoblast layer.

207. The composition of claim 204, wherein the second birth tissue is selected from the group consisting of an umbilical cord, an amniotic sac, a placental plate, a placental membrane and a combination thereof, wherein the placental membrane is from the amniotic sac and comprises amniotic membrane, chorionic membrane and trophoblast layer.

208. The composition of claim 203, wherein the composition is injectable.

209. The composition of claim 204, wherein the composition has a shear viscosity of 0.1-500 Pa·s at 0.5 Hz.

210. The composition of claim 203, further comprising less than 5 mg/ml solubilized collagen and/or solubilized laminin.

211. The composition of claim 203, further comprising one or more bioactive factors selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, and HA.

212. The composition of claim 203, further comprising PRG-4 at a concentration greater than 0.2 ng/MI, α2-Macroglobulin at a concentration greater than 4 μg/mL, TGF-beta3 at a concentration at least 0.5 ng/mL, or any combination thereof.

213. The composition of claim 203, further comprising hyaluronic acid (HA) not from the first birth tissue.

214. The composition of claim 203, wherein the body part is a joint or tissue.

215. The composition of claim 214, wherein the joint is selected from the group consisting of knee, shoulder, hip, elbow, wrist, finger, toe and ankle joints.

216. The composition of claim 214, wherein the tissue is selected from the group consisting of tendon, ligament, bursa, fascia, cartilage, muscle, connective tissue, dermis, synovium, and enthesis.

217. The composition of claim 203, wherein the pathological condition is selected from the group consisting of osteoarthritis, rheumatoid arthritis, bursitis, fasciitis, tendonitis, tendinopathy, synovitis, epicondylitis, tendon rupture, ligament rapture, nerve damage, cartilage defect, synovitis, fasciitis pain, arthroplasty, and muscle pain.

218. A method for treating a pathological condition in a body part of a patient in need thereof, comprising administering to the body part of the patient an effective amount of the composition of claim 203.

219. The method of claim 218, wherein the body part is a joint or tissue, and the joint is selected from the group consisting of knee, shoulder, hip, elbow, wrist, finger, toe and ankle joints.

220. A composition comprising a soluble portion and a solid portion, wherein the soluble portion comprises an elute of a first birth tissue and the solid portion comprises particulates of a second birth tissue, wherein the soluble portion and the solid portion each comprise one or more bioactive factors selected from the group consisting of HGF, IL-IRA, PTX-3, IL-8, G-CSF, MCP1, TIMP-1, TIMP-2, TIMP-3, TIMP-4, α2-Macroglobulin, bFGF, PIGF, EGF, TGF-beta1, TGF-beta2, TGF-beta3, PDGF-BB, VEGF-α, Angiogenin, PRG-4, HA, extracellular vesicles and exosomes.

221. A method for providing one or more bioactive factors to a body part of a patient in need thereof, comprising administering to the body part of the patient an effective amount of a composition of claim 220, wherein the soluble portion and the solid portion each comprise one or more bioactive factors, and optionally releasing 5-50% of the one or more bioactive factors to the body part within 1 minute after the administration.

222. The method of claim 221, wherein the one or more bioactive factors are selected from the group consisting of TIMP-3, PRG-4, TGF-beta3, α2-Macroglobulin, and combinations thereof.
